(19) United States
(12) Patent Application Publication
(10) Pub. No.: US 2008/0261898 A1
(43) Pub. Date: Oct. 23, 2008

(54) COMPOSITION AND METHOD FOR CANCER TREATMENT AND PREVENTION

(76) Inventors: Heather H. Liao, Los Altos, CA (US); Li Zhan, Beijing (CN)

Correspondence Address: WILSON SONSINI GOODRICH & ROSATI, 650 PAGE MILL ROAD, PALO ALTO, CA, 94304-1050, US

(21) Appl. No.: 12/105,173

(22) Filed: Apr. 17, 2008

Related U.S. Application Data
(60) Provisional application No. 60/912,367, filed on Apr. 17, 2007.

(51) Int. Cl. A61K 31/7048; A61K 31/352; C07D 311/30; A61P 35/00; C07H 17/07

(52) U.S. Cl. 514/25; 549/403; 514/456; 536/8

(57) ABSTRACT

Compounds, compositions, methods and kits are provided for treating, reducing the risk of, or preventing diseases and/or conditions, such as diseases associated with angiogenesis and/or abnormal cell proliferation, such as cancer. Compounds of the present invention have antiangiogenic and anti-cancer activity with minimum toxic effects on normal cells. Methods for preparing and manufacturing the compounds and pharmaceutical compositions are also provided.

### RELATED PATENT APPLICATIONS

This application claims priority to U.S. provisional application No. 60/912,367 filed Apr. 17, 2007, the contents of which are hereby incorporated by reference in the entirety.

### BACKGROUND OF THE INVENTION

Angiogenesis, the cell cycle, and apoptosis are under homeostatic control. Dysregulation of these processes is causative or associated with numerous diseases and conditions. Dysregulated angiogenesis is involved in diabetic retinopathy, neovascular glaucoma, psoriasis, retrolental fibroplasias, angiofibroma, and inflammation. Additionally, cancerous and neoplastic tissues recruit neovasculature to supply nutrients to growing tumor masses. This neovasculature can also serve as a route for cancer cells to enter the circulatory system and travel to distance sites in the body leading to metastases.

Dysregulation of the cell cycle that allows for uncontrolled cell proliferation is involved in growth of benign and malignant tumors, including tumor metastasis, hematologic malignancies, restenosis, abnormal stimulation of endothelial cells (atherosclerosis), insults to body tissue due to surgery (surgical adhesions). Dysregulation of apoptosis where cells do not undergo programmed cell death in response to stimuli or DNA damage results in an abnormal accumulation of cells such as a tumor.

Current therapy of diseases or conditions that involve dysregulated angiogenesis, cell cycle control or apoptosis is ineffective. There remains a need for improved therapeutic methods. The methods and compositions of the present invention provide the basis for pharmaceutical compositions useful in the prevention and treatment of diseases and conditions associated with dysregulated angiogenesis, cell cycle control and apoptosis as well as for other needs.

### INCORPORATION BY REFERENCE

All publications and patent applications mentioned in this specification are herein incorporated by reference to the same extent as if each individual publication or patent application was specifically and individually indicated to be incorporated by reference.

### BRIEF SUMMARY OF THE INVENTION

The present invention relates to novel compounds, compositions, methods, dose unit forms, and kits for treating or preventing diseases and/or conditions, such as diseases associated with angiogenesis and abnormal cell proliferation. More particularly, the present invention relates to the treatment or prevention of cancer by selective inhibition of cancer cell and tumor mass proliferation and growth mechanisms that demonstrate minimal adverse effects on normal cell growth.

In one aspect of the invention, a compound of Formula I, its pharmaceutically acceptable salt, ester, prodrug, stereisomer or tautomer, is provided:

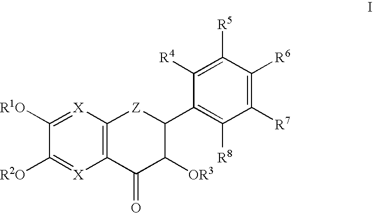

wherein

X is independently CH or N;

Z is independently O, S or NH;

R₁, R₂ and R₃ are independently selected from the group consisting of hydrogen, substituted or unsubstituted C₁-C₁₀ alkyl, substituted or unsubstituted C₁-C₁₀ alkenyl, substituted or unsubstituted C₁-C₁₀ alkynyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether and carbohydrate; and

R₄, R₅, R₆, R₇, R₈ are independently selected from the group consisting of hydrogen, substituted or unsubstituted hydroxyl, substituted or unsubstituted amine, substituted or unsubstituted thiol, substituted or unsubstituted C₁-C₁₀ alkyl, substituted or unsubstituted C₁-C₁₀ alkynyl, substituted or unsubstituted C₁-C₁₀ alkenyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether and carbohydrate.

In some embodiments, X is CH; Z is O; and R₁ and R₂ are independently selected from the group consisting of hydrogen, substituted or unsubstituted C₁-C₁₀alkyl, substituted or unsubstituted C₁-C₁₀ aliphatic acyl and substituted or unsubstituted C₁-C₁₀ aromatic acyl.

In some embodiments, X is CH; Z is O; R₁ and R₂ are independently selected from the group consisting of hydrogen and substituted or unsubstituted C₁-C₁₀ aliphatic acyl; and R₄, R₅, R₆, R₇, R₈ are independently selected from the group consisting of hydrogen, substituted or unsubstituted hydroxyl, substituted or unsubstituted amine, and substituted or unsubstituted C₁-C₁₀ aliphatic acyl. Preferably, R₄, R₅, R₆, R₇, R₈ are independently selected from the group consisting of substituted or unsubstituted hydroxyl and substituted or unsubstituted amine.

Optionally, at least three of R₄, R₅, R₆, R₇, R₈ are hydrogen. Preferably, R₅ and R₆ are substituted or unsubstituted hydroxyl and R₄, R₇, R₈ are hydrogen.

Optionally, R₃ is independently selected from the group consisting of hydrogen, substituted or unsubstituted C₁-C₁₀ alkyl and carbohydrate. Preferably, R₃ is H or carbohydrate. The carbohydrate may be a monosaccharide, a disaccharide or a polysaccharide.

In one aspect of the invention, a compound of Formula II, its pharmaceutically acceptable salt, ester, prodrug, stereisomer or tautomer, is provided:

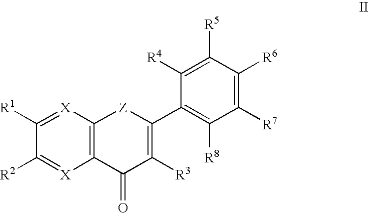

wherein

X is independently CH or N;

Z is independently O, S or NH,

R¹, R², and R³ are each, independently -L-R⁹,

L is —O—, —OC(═O)—, —OP(O)₀₋₁(OR¹⁰)O—, —P(O)₀₋₁(OR¹⁰)O—, —S—, —S(O)—, —S(O)₂—, —S(O)₂NR¹⁰—, or —NR¹⁰—,

R⁹ and R¹⁰ are each independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R¹¹ substituents,

R¹¹ is halogen, —OR², —SH, NH₂, —NR¹²R¹³, —CO₂R¹², —CO₂aryl-C(═O)NR¹²R¹³, —NO₂, —CN, —S(O)₀₋₂C₁₋₁₀alkyl, —S(O)₀₋₂aryl, —SO₂NR¹²R¹³, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR⁹R¹⁰, —SO₂ NR¹²R¹³, or —NR¹²R¹³ substituents,

R⁴, R⁵, R⁶, R⁷ and R³ are each independently hydrogen, halogen, —OH, —R¹², —OR¹², —SH, NH₂, —NR¹²R¹³, —CO₂R¹², —CO₂aryl-C(═O)NR¹²R¹³, —NO₂, —CN, —S(O)₀₋₂C₁₋₁₀alkyl, —S(O)₀₋₂aryl, —SO₂NR¹²R¹³, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR¹²R¹³, —SO₂NR¹²R¹³, —SO₂NR¹²R¹³, —NR¹²R¹³, or trialkylsilyl substituents,

R¹² and R¹³ in each instance, are independently H or unsubstituted or substituted C₁₋₁₀alkyl with one or more aryl, heteroalkyl, heterocyclyl, or hetaryl substituents, wherein each of said alkyl, aryl, heteroalkyl, heterocyclyl, or hetaryl groups is unsubstituted or substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂ N(C₁₋₁₀alkyl)(C₁₋₁₀alkyl), or —SO₂NH(C₁₋₁₀alkyl) substituents.

In some embodiments X is CH; Z is O; L is —O—; R¹, R², and R³, each R⁹ is independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R¹¹ substituents; and R⁴, R⁵, R⁶, R⁷ and R⁸ are each independently selected from the group of hydrogen, —OH, —R¹², —OR¹², NH₂, and —NR¹²R¹³. In some embodiments at least three of R⁴, R⁵, R⁶, R⁷ and R⁸ are hydrogen. In some embodiments R⁴, R⁷ and R⁸ are hydrogen, and R⁵ and R⁶ are each independently selected from the group of hydrogen, —OH and —OR¹². In some embodiments R³ is —OH, —OR¹², or —O-carbohydrate. In other embodiments R³ is —OH or —O-carbohydrate. In some embodiments the carbohydrate is a monosaccharide, a disaccharide, or a polysaccharide. In other embodiments the monosaccharide is selected from the group consisting of glucose, galactose, and fructose. In additional embodiments the disaccharide is selected from the group consisting of sucrose, lactose, maltose and rutinose. In further embodiments, the polysaccharide is selected from the group consisting of starch, glycogen, and cellulose.

In one aspect of the invention, a compound of Formula III, its pharmaceutically acceptable salt, ester, prodrug, stereoisomer or tautomer is provided:

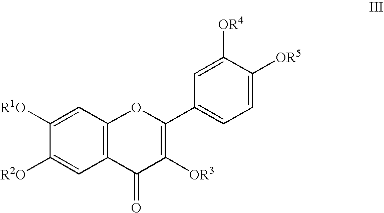

wherein R¹, R², R³, R⁴, and R⁵ are each independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R⁶ substituents,

R⁶ is halogen, —OR⁷, —SH, NH₂, —NR⁷R⁸, —CO₂R⁷, —CO₂aryl, —C(═O)NR⁷R⁸, —NO₂, —CN, —S(O)₀₋₂C₁₋₁₀alkyl, —S(O)₀₋₂aryl, —SO₂NR⁷R⁸, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR⁷R⁸, —SO₂ NR⁷R⁸, or —NR⁷R⁸ substituents,

R⁷ and R⁸ are independently H or unsubstituted or substituted C₁₋₁₀alkyl with one or more aryl, heteroalkyl, heterocyclyl, or hetaryl substituents, wherein each of said alkyl, aryl, heteroalkyl, heterocyclyl, or hetaryl groups is unsubstituted or substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl) or substituents.

In some embodiments R³ is hydrogen or carbohydrate. In other embodiments, R⁴ and R⁵ are each independently hydrogen, C₁₋₁₀alkyl, or carbohydrate, each of which except for hydrogen is unsubstituted or substituted by one or more independent R⁶ substituents. In some embodiments R¹ and R² are each independently hydrogen, C₁₋₁₀alkyl, or carbohydrate, each of which except for hydrogen is unsubstituted or substituted by one or more independent R⁶ substituents. In other embodiments R¹, R², R⁴, and R⁵ are each independently hydrogen, unsubstituted C₁₋₁₀alkyl, or carbohydrate. In some embodiments R¹, R², R⁴, and R⁵ are each independently hydrogen or unsubstituted C₁₋₁₀alkyl, and R³ is hydrogen or carbohydrate. In some embodiments the carbohydrate is a monosaccharide, a disaccharide, or a polysaccharide. In other embodiments, the monosaccharide is selected from the group consisting of glucose, galactose, and fructose. In some embodiments, the disaccharide is selected from the group consisting of sucrose, lactose, maltose and rutinose. In other embodiments, the polysaccharide is selected from the group consisting of starch, glycogen, and cellulose.

In some embodiments, R¹, R², R⁴, and R⁵ are each hydrogen, and R³ is hydrogen or carbohydrate. In other embodiments, R³ is hydrogen or carbohydrate. In some embodiments, R³ is carbohydrate. In some embodiments R³ is rutinose. In one embodiment, the compound of Formula III is AC620 and has the following structure:

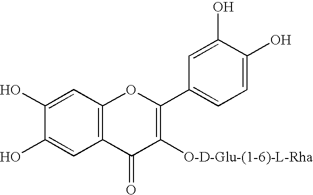

In one aspect of the invention, a pharmaceutical composition comprising a compound of Formula I, II, or III, its pharmaceutically acceptable salt, ester, prodrug, stereoisomer or tautomer; and a pharmaceutically acceptable carrier is provided.

In one aspect, a method of treating, reducing the risk of, or preventing cancer in a mammal, comprising administering to a subject in need thereof an effective amount of a pharmaceutical composition comprising the compound of Formula I, II, or III, its pharmaceutically acceptable salt, ester, prodrug, stereoisomer or tautomer.

In one embodiment, the cancer is selected from the group consisting of adrenal cortical cancer, anal cancer, bile duct cancer, bladder cancer, bone cancer, bone metastasis, adult CNS brain tumors, children CNS brain tumors, breast cancer, Castleman's Disease, cervical cancer, childhood non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, glioblastoma, lymphomas, renal cell cancer, and head and neck cancer. In a further embodiment, treatment further comprises treating the subject with surgery, radiation therapy, chemotherapy, gene therapy, immunotherapy, or a combination thereof.

In another embodiment, the pharmaceutical composition is administered to the mammal orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, transurethrally, topically, or via an implanted reservoir. In one embodiment, the mammal is a human.

In one aspect, a method of treating or preventing a disease in which modulation of angiogenesis is desirable in a mammal is provided, that comprises administering to a subject in need thereof an effective amount of a pharmaceutical composition comprising the compound of Formula I, II, or III, its pharmaceutically acceptable salt, ester, prodrug, stereoisomer or tautomer.

In one embodiment, the disease in which modulation of angiogenesis is desirable is selected from the group consisting of rheumatoid arthritis, psoriasis, atherosclerosis, diabetic and other retinopathies, retrolentral fibroplasia, neovascular glaucoma, age-related macular degeneration, thyroid hyperplasias, grave's disease, tissue transplantation, chronic inflammation, lung inflammation, nephrotic syndrome, preclampasia, ascites, pericardial effusion, pleural effusion, coronary artery disease, and peripheral artery disease.

In another embodiment, the pharmaceutical composition is administered to the mammal orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, transurethrally, topically, or via an implanted reservoir. In a further embodiment, the mammal is a human.

In one aspect of the invention, a pharmaceutical composition in unit dosage form is provided comprising, per dosage unit, an amount of a compound of Formula I, II, or III, or a pharmaceutically acceptable salt, ester, prodrug, stereoisomer or tautomer thereof within the range from about 1 mg to about 10 g; and a pharmaceutically acceptable carrier, diluent or excipient. In one embodiment, the amount of a compound of Formula I, II, or III, or a pharmaceutically acceptable salt, ester, prodrug, stereoisomer or tautomer is within the range from about 10 mg to about 500 mg. In another embodiment, the amount of a compound of Formula I, II, or III, or a pharmaceutically acceptable salt, ester, prodrug, stereoisomer or tautomer is within the range from about 10 mg to about 100 mg.

In one aspect of the invention, a kit is provided comprising a container or vessel comprising the compound of Formula I, II, or III, its pharmaceutically acceptable salt, ester, prodrug, stereoisomer or tautomer. In one embodiment the container or vessel comprises a pharmaceutical composition comprising the compound of Formula I, II, or III, its pharmaceutically acceptable salt, ester, prodrug, stereoisomer or tautomer. In another embodiment, the kit further comprises written instructions for using said pharmaceutical composition for treating or preventing said disease or condition. In a further embodiment, the disease or condition is selected from the group consisting of adrenal cortical cancer, anal cancer, bile duct cancer, bladder cancer, bone cancer, bone metastasis, adult CNS brain tumors, children CNS brain tumors, breast cancer, Castleman Disease, cervical cancer, Childhood Non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, glioblastoma, lymphomas, renal cell cancer, and head and neck cancer.

In another embodiment, the disease or condition is selected from the group consisting of rheumatoid arthritis, psoriasis, atherosclerosis, diabetic and other retinopathies, retrolentral fibroplasia, neovascular glaucoma, age-related macular degeneration, thyroid hyperplasias, grave's disease, tissue transplantation, chronic inflammation, lung inflammation, nephrotic syndrome, preclampasia, ascites, pericardial effusion, pleural effusion, coronary artery disease, and peripheral artery disease.

### BRIEF DESCRIPTION OF THE DRAWINGS

Aspects of the invention can be more fully understood with respect to the following drawings.

FIG. 1 illustrates an exemplary method of synthesis for Compound 6.

FIG. 2 illustrates an exemplary method of synthesis for Compound 10.

FIG. 3A illustrates an exemplary method of synthesis for Compound 20.

FIG. 3B illustrates an exemplary method of synthesis for Compound 22 (AC620).

FIG. 4 illustrates the nuclear magnetic resonance spectroscopy (NMR) of AC620: full spectrum ¹H-NMR spectrograph

FIG. 5 illustrates the NMR spectroscopy of AC620: partial amplified ¹H-NMR spectrograph: δ 0.85-1.35 ppm

FIG. 6 illustrates the NMR spectroscopy of AC620: partial amplified ¹H-NMR spectrograph: δ 3.1-4.0 ppm

FIG. 7 illustrates the NMR spectroscopy of AC620: partial amplified ¹H-NMR spectrograph: δ 4.3-5.3 ppm

FIG. 8 illustrates the NMR spectroscopy of AC620: partial amplified ¹H-NMR spectrograph: δ 6.1-7.6 ppm

FIG. 9 illustrates the NMR spectroscopy of AC620: full spectrum ¹³H-NMR spectrograph

FIG. 10 illustrates the NMR spectroscopy of AC620: partial amplified ¹³H-NMR spectrograph: δ 60-80 ppm

FIG. 11 illustrates the NMR spectroscopy of AC620: partial amplified ¹³H-NMR spectrograph: δ 90-125 ppm

FIG. 12 illustrates the NMR spectroscopy of AC620: partial amplified ¹³H-NMR spectrograph: δ 130-180 ppm

FIG. 13 illustrates the fast atom bombardment mass spectroscopy (FAB-MS) of AC620

FIG. 14 illustrates the effect of AC620 on VEGF expression in DU-145 cells, a metastatic prostate cancer cell line.

FIG. 15 illustrates the effect of AC620 on VEGF R1 and VEGF R2 expression in DU-145 cells.

FIG. 16 illustrates the effect of AC620 on cell proliferation of DU-145 cells at various concentrations.

FIG. 17 illustrates the effect of AC620 on cell proliferation of LNCaP cells, a metastatic prostate cancer cell line.

FIG. 18 illustrates the effect of AC620 on cell proliferation of DU-145 cells at various time points.

FIG. 19 illustrates the effect of AC620 on cell proliferation of LNCaP cells at various time points.

FIG. 20 illustrates the effect of AC620 on cell cycle in DU-145 cells.

FIG. 21 illustrates the effect of AC620 on CDK2 expression in DU-145 cells.

FIG. 22 illustrates the effect of AC620 on apoptosis in DU-145 cells.

FIG. 23 illustrates the effect of AC620 on apoptosis in LNCaP cells.

FIG. 24 illustrates the effect of AC620 on cell viability in DU-145 cells, LNCaP cells and primary human prostate endothelial cells.

FIG. 25 illustrates the effect of AC620 on cell proliferation of HT-29 cells, a metastatic colorectal cancer cell line.

FIG. 26 illustrates the effect of AC620 on apoptosis in HT-29 cells.

FIG. 27 illustrates the effect of AC620 on tumor growth in a xenograft mouse model of human prostate cancer.

FIG. 28 illustrates the effect of AC620 on VEGF and VEGF R2 expression in tumor samples from a xenograft mouse model.

FIG. 29 illustrates the effect of AC620 on VEGF RNA expression in tumor samples from a xenograft mouse model.

FIG. 30 illustrates the effect of AC620 on CDK1 and CDK2 expression in tumor samples from a xenograft mouse model.

FIG. 31 illustrates the effect of AC620 on Bcl-2 and p21 expression in tumor samples from a xenograft mouse model.

### DEFINITIONS

The term “inhibiting” or “inhibition,” as used herein, refers to any detectable negative effect on gene expression, cell proliferation or tumor growth. Such a negative effect may include the slowing or arrest of cell proliferation as well as the induction of cell death. Typically, an inhibition is reflected in a decrease of at least 10%, more preferably at least 20%, 30%, 40%, 50%, 60%, 70%, 80%, 90%, or 95% in gene expression or cell proliferation when compared to a control.

An inhibition in cell proliferation may also be measured by an increase in apoptosis. Typically, increased apoptosis is reflected by an increase of at least 10%, more preferably at least 20%, 30%, 40%, 50%, 60%, 70%, 80%, 90%, or 95% compared to a control.

The term “effective amount,” as used herein, refers to an amount that produces therapeutic effects for which a substance is administered. The effects include the prevention, correction, or inhibition of progression of the symptoms of a disease/condition and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of pharmaceutical Compounding (1999); and Pickar, Dosage Calculations (1999)).

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention

As used herein, for example, “C₁₋₄ alkyl” is used to mean an alkyl having 1-4 carbons—that is, 1, 2, 3, or 4 carbons in a straight or branched configuration. In all embodiments of this invention, the term “alkyl” includes both branched and straight chain alkyl groups, having the number of carbon atoms designated (i.e. C₁₋₁₀ means one to ten carbons and C₂₋₁₀ means two to ten carbons). Typical saturated alkyl groups are methyl, ethyl, n-propyl, isopropyl, n-butyl, sec-butyl, isobutyl, tert-butyl, n-pentyl, isopentyl, n-hexyl, n-heptyl, isooctyl, nonyl, decyl, undecyl, dodecyl, tetradecyl, hexadecyl, octadecyl, eicosyl, and the like.

The term “halo” or “halogen” refers to fluoro, chloro, bromo, or iodo.

The term “acyl” refers to the structure —C(═O)—R, in which R is a general substituent variable such as, for example for the R groups described in the Formulae above. Examples include, but are not limited to, (bi)(cyclo)alkylketo, (cyclo)alkenylketo, alkynylketo, arylketo, hetarylketo, heterocyclylketo, heterobicycloalkylketo, spiroalkylketo.

Unless otherwise specified, the term “cycloalkyl” refers to a 3-8 carbon cyclic aliphatic ring structure, optionally substituted with for example, alkyl, hydroxy, oxo, and halo, such as cyclopropyl, methylcyclopropyl, cyclobutyl, cyclopentyl, 2-hydroxycyclopentyl, cyclohexyl, 4-chlorocyclohexyl, cycloheptyl, cyclooctyl, and the like.

As used herein, the term “heteroatom” or “ring heteroatom” is meant to include oxygen (O), nitrogen (N), sulfur (S), phosphorus (P), and silicon (Si).

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)NR′, —NR′R″, —OR′, —SR′, and/or —SO2R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “alkenyl” refers to an ethylenically unsaturated hydrocarbon group, straight or branched chain, having 1 or 2 ethylenic bonds, for example vinyl, allyl, 1-butenyl, 2-butenyl, isopropenyl, 2-pentenyl, and the like.

The term “alkynyl” refers to an unsaturated hydrocarbon group, straight or branched, having at least one acetylenic bond, for example ethynyl, propargyl, and the like.

The term “aryl” refers an aromatic ring such as phenyl or naphthyl which may be optionally substituted. Examples of aryl include, but are not limited to, phenyl, 4-chlorophenyl, 4-fluorophenyl, 4-bromophenyl, 3-nitrophenyl, 2-methoxyphenyl, 2-methylphenyl, 3-methylphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, and 2-iodo-4-methylphenyl.

The terms “heteroaryl” or “hetaryl” or “heteroar-” or “hetar-” refer to a substituted or unsubstituted 5- or 6-membered unsaturated ring containing one, two, three, or four independently selected heteroatoms, preferably one or two heteroatoms independently selected from oxygen, nitrogen, and sulfur or to a bicyclic unsaturated ring system containing up to 10 atoms including at least one heteroatom selected from oxygen, nitrogen, and sulfur. Examples of hetaryls include, but are not limited to, 2-, 3- or 4-pyridinyl, pyrazinyl, 2-, 4-, or 5-pyrimidinyl, pyridazinyl, triazolyl, tetrazolyl, imidazolyl, 2- or 3-thienyl, 2- or 3-furyl, pyrrolyl, oxazolyl, isoxazolyl, thiazolyl, isothiazolyl, oxadiazolyl, thiadiazolyl, quinolyl, isoquinolyl, benzimidazolyl, benzotriazolyl, benzofuranyl, and benzothienyl. The heterocyclic ring may be optionally substituted with one or more substituents.

The terms “aryl-alkyl” or “arylalkyl” or “aralkyl” are used to describe a group wherein the alkyl chain can be branched or straight chain forming a bridging portion with the terminal aryl, as defined above, of the aryl-alkyl moiety. Examples of aryl-alkyl groups include, but are not limited to, optionally substituted benzyl, phenethyl, phenpropyl and phenbutyl such as 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitrophenyl)ethyl, 2-(2,4-dichlorophenyl)ethyl, 2-(3,5-dimethoxyphenyl)ethyl, 3-phenylpropyl, 3-(3-chlorophenyl)propyl, 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxphenyl)butyl, and 10-phenyldecyl.

The terms “hetarylalkyl” or “heteroarylalkyl” or “hetaryl-alkyl” or “heteroaryl-alkyl” or “hetaralkyl” or “heteroaralkyl” are used to describe a group wherein the alkyl chain can be branched or straight chain forming a bridging portion of the heteroaralkyl moiety with the terminal heteroaryl portion, as defined above, for example 3-furylmethyl, thenyl, furfuryl, and the like.

The term “hetaryl-C₁₋₁₀ alkyl” is used to describe a hetaryl alkyl group as described above where the alkyl group contains 1 to 10 carbon atoms.

The terms “hetarylalkenyl” or “heteroarylalkenyl” or “hetaryl-alkenyl” or “heteroaryl-alkenyl” or “hetaralkenyl” or “heteroaralkenyl” are used to describe a hetarylalkenyl group wherein the alkenyl chain can be branched or straight chain forming a bridging portion of the heteroaralkenyl moiety with the terminal heteroaryl portion, as defined above, for example 3-(4-pyridyl)-1-propenyl.

The term “hetaryl-C₂₋₁₀ alkenyl” group is used to describe a group as described above wherein the alkenyl group contains 2 to 10 carbon atoms.

The terms “hetarylalkynyl” or “heteroarylalkynyl” or “hetaryl-alkynyl” or “heteroaryl-alkynyl” or “hetaralkynyl” or “heteroaralkynyl” are used to describe a group wherein the alkynyl chain can be branched or straight chain forming a bridging portion of the heteroaralkynyl moiety with the heteroaryl portion, as defined above, for example 4-(2-thienyl)-1-butynyl, and the like.

The term “hetaryl-C₂₋₁₀ alkynyl” is used to describe a hetarylalkynyl group as described above wherein the alkynyl group contains 2 to 10 carbon atoms.

The term “heterocyclyl” or “hetcyclyl” refers to a substituted or unsubstituted 3-, 4-, 5-, or 6-membered saturated or partially unsaturated ring containing one, two, or three heteroatoms, preferably one or two heteroatoms independently selected from oxygen, nitrogen and sulfur; or to a bicyclic ring system containing up to 10 atoms including at least one heteroatom independently selected from oxygen, nitrogen, and sulfur wherein the ring containing the heteroatom is saturated. Examples of heterocyclyls include, but are not limited to, tetrahydrofuranyl, tetrahydrofuryl, pyrrolidinyl, piperidinyl, 4-pyranyl, tetrahydropyranyl, thiolanyl, morpholinyl, piperazinyl, dioxolanyl, dioxanyl, indolinyl, and 5-methyl-6-chromanyl.

Compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above compounds of Formula I, II, or III are shown without a definitive stereochemistry at certain positions. The present invention includes all stereoisomers of Formula I, II, or III and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The present invention includes all manner of rotamers and conformationally restricted states of a compound of the invention.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present.

When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl, 4 piperazinyl, and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are selected from, for example: halogen, —OR′, —NR′R″, —SR′, -halogen, —SiR′R″R′″, —OC(O)R′, —C(O)R′, —CO2R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N3, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Examples of monosaccharide include, but are not limited to, glucose, galactose, and fructose with their structures shown below.

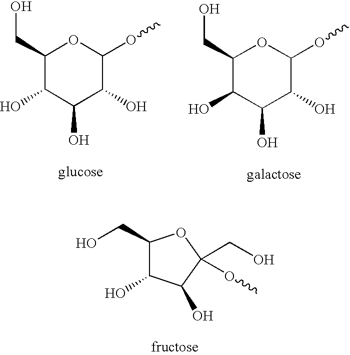

Examples of disaccharide include, but are not limited to, sucrose, lactose and maltose with their structures shown below, and rutinose (6-O-L-rhamnosyl-D-glucose).

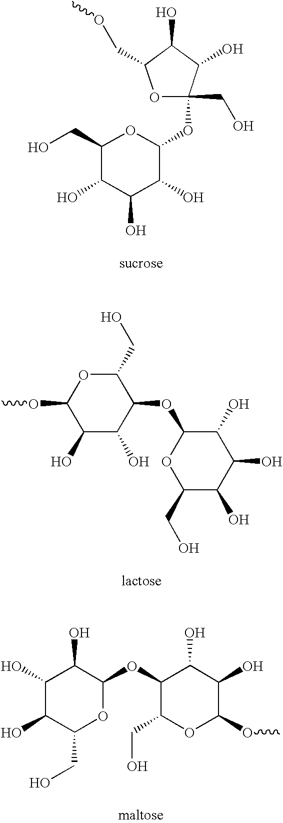

Examples of polysaccharide include, but are not limited to, starch, glycogen, and cellulose.

### DETAILED DESCRIPTION OF THE INVENTION

The term angiogenesis refers to the generation or formation of new blood vessels in a tissue or organ. Angiogenesis occurs during some normal physiological processes and is also present in some pathological conditions. The process of angiogenesis involves endothelial cells that form the inner lining of the blood vessels. In response to stimuli, endothelial cells proliferate and release enzymes that erode the basement membrane through which the endothelial cells cause protrusions. These cells can then migrate through the protrusions and form a sprout of the parent blood vessel. The merging of endothelial cell sprouts forms capillary loops leading to the formation of new blood vessels. Examples of angiogenesis during normal physiological processes include wound healing, blastcyst implantation and fetal growth, and the repair of the corpus luteum, and endometrium. Pathological states associated with angiogenesis include diabetic retinopathy, retrolental fibroplasia, corneal graft neovascularization, neovascular glaucoma, trachoma, psoriasis and pyogenic granuloma, hemangioma, angiofibroma, hemophiliac joints, hypertrophic scars, wound granulation, vascular adhesions, rheumatoid arthritis, scleroderma; atherosclerotic plaque and cancer.

In cancer, the new blood vessels support the continued growth of tumors by providing nutrients. The induced new tumor blood supply also provide tumor cells access to the vascular system where tumor cells can then travel to distant sites and form metastases.

The development of methods for the selective prevention, reduction, or cession of angiogenesis may be of benefit in the aforementioned pathological conditions. Additionally, compounds of the present invention may serve as birth control agents by preventing vascularization required for blastocyst implantation and also for development of the placenta, blastcyst, embryo and or fetus.

The cell cycle is a series of essential biochemical events involving DNA replication and segregation that take place in a eukaryotic cell as it replicates. Compounds that modulate or inhibit the progression of cells through the cell cycle may provide new treatments for diseases and conditions characterized by excessive or pathological cell proliferation, including cancer.

One target for modulation or inhibition of activity is cyclin-dependent kinases (CDK) such as CDK1 and CDK2. Growth signals cause quiescent, or G₀ state to enter the cell cycle at G₁ phase. CDK2 associates with cyclin E to drive cells from G1 to S phase. On entry into S phase, cyclin E is abruptly destroyed and cyclin A, expressed in response to the CDK2/cyclin E activities, then associates with CDK2 to drive cells through S phase. CDK1 is required for the proper segregation of cellular material between daughter cells during cell division.

Another target for modulation or inhibition is p21, a facilitator of the assembly of D-type cyclins with CDK4 and CDK6. p21 also directs the translocation of the assembled complexes to the nucleus and prevents their nuclear export. This results in elevated levels of active D-type cyclin/CDKs that initiate Retinoblastoma protein phosphorylation, thereby promoting progression through the G1 phase of the cell cycle. Similarly, cyclin B/CDK1 activity is activated in a p21-dependent manner at the G2/M transition.

The induction of apoptosis, also known as programmed cell death, is another way to target cancer cells as they are generally resistant to signals to undergo apoptosis and instead continue to live and proliferate inappropriately. One target for inhibition or modulation is Bcl-2, a pro-survival apoptosis regulator found on the outer membranes of the mitochondria. Over-expression of Bcl-2 makes cells resistant to pro-apoptotic stimuli. p21 is also involved in apoptosis where it binds to and prevents the activation of procaspase 3, a necessary component of the Fas-mediated apoptotic pathway. p21 further inhibits the activity of the pro-apoptotic kinase ASK1. Treatment with inhibitors or modulators of Bcl-2 and/or p21, may therefore exert a synergist effect on cancer and other diseases or conditions that feature pathological cell proliferation.

The present invention relates to novel compounds, compositions, methods, unit dose forms and kits for treating or preventing diseases and/or conditions, associated with abnormal cell proliferation and angiogenesis. More particularly, the present invention provides means for the selective inhibition of angiogenesis, the promotion of cell cycle arrest, and the induction of apoptosis with minimum adverse effects on normal cell growth that are useful in the treatment of cancer and other diseases and conditions.

In one aspect of the invention, a compound of Formula I, its pharmaceutically acceptable salt, ester, prodrug, stereisomer or tautomer, is provided:

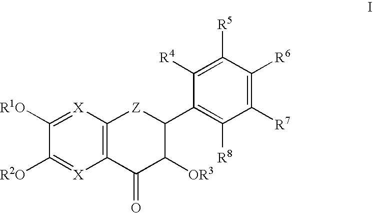

wherein

X is independently CH or N;

Z is independently O, S or NH;

R₁, R₂ and R₃ are independently selected from the group consisting of hydrogen, substituted or unsubstituted C₁-C₁₀ alkyl, substituted or unsubstituted C₁-C₁₀ alkenyl, substituted or unsubstituted C₁-C₁₀ alkynyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether and carbohydrate; and

R₄, R₅, R₆, R₇, R₈ are independently selected from the group consisting of hydrogen, substituted or unsubstituted hydroxyl, substituted or unsubstituted amine, substituted or unsubstituted thiol, substituted or unsubstituted C₁-C₁₀ alkyl, substituted or unsubstituted C₁-C₁₀ alkynyl, substituted or unsubstituted C₁-C₁₀ alkenyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether and carbohydrate.

In some embodiments, X is CH; Z is O; and R₁ and R₂ are independently selected from the group consisting of hydrogen, substituted or unsubstituted C₁-C₁₀ alkyl, substituted or unsubstituted C₁-C₁₀ aliphatic acyl and substituted or unsubstituted C₁-C₁₀ aromatic acyl.

In some embodiments, X is CH; Z is O; R₁ and R₂ are independently selected from the group consisting of hydrogen and substituted or unsubstituted C₁-C₁₀ aliphatic acyl; and R₄, R₅, R₆, R₇, R₈ are independently selected from the group consisting of hydrogen, substituted or unsubstituted hydroxyl, substituted or unsubstituted amine, and substituted or unsubstituted C₁-C₁₀ aliphatic acyl. Preferably, R₄, R₅, R₆, R₇, R₈ are independently selected from the group consisting of substituted or unsubstituted hydroxyl and substituted or unsubstituted amine.

Optionally, at least three of R₄, R₅, R₆, R₇, R₈ are hydrogen. Preferably, R₅ and R₆ are substituted or unsubstituted hydroxyl and R₄, R₇, R₈ are hydrogen.

Optionally, R₃ is independently selected from the group consisting of hydrogen, substituted or unsubstituted C₁-C₁₀ alkyl and carbohydrate. Preferably, R₃ is H or carbohydrate. The carbohydrate may be a monosaccharide, a disaccharide or a polysaccharide.

In one aspect of the invention, a compound of Formula II, its pharmaceutically acceptable salt, ester, prodrug, stereisomer or tautomer, is provided:

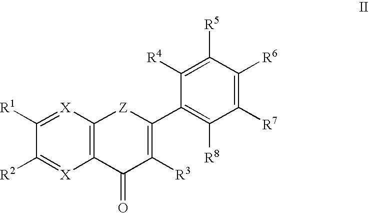

wherein

X is independently CH or N;

Z is independently O, S or NH,

R¹, R², and R³ are each, independently -L-R⁹,

L is —O—, —OC(═O)—, —OP(O)₀₋₁(OR¹⁰)O—, —P(O)₀₋₁(OR¹⁰)O—, —S—, —S(O)—, —S(O)₂—, —S(O)₂NR¹⁰—, or —NR¹⁰,

R⁹ and R¹⁰ are each independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R¹¹ substituents,

R¹¹ is halogen, —OR², —SH, NH₂, —NR¹²R¹³, —CO₂R¹², —CO₂aryl —C(═O)NR¹²R¹³, —NO₂, —CN, —S(O)₀₋₂C₁₋₁₀alkyl, —S(O) 0 ₂aryl, —SO₂NR¹²R¹³, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR⁹R¹⁰, —SO, NR¹²R¹³, or —NR¹²R¹³ substituents,

R⁴, R⁵, R⁶, R⁷ and R⁸ are each independently hydrogen, halogen, —OH, —R², —OR¹², —SH, NH₂, —NR¹²R¹³, —CO₂R¹², —CO₂aryl —C(═O)NR¹²R¹³, —NO₂, —CN, —S(O)₀₋₂C₁₋₁₀alkyl, —S(O)₀₋₂aryl, —SO₂NR¹²R¹²R¹³, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(—O)NR¹²R¹³, —SO₂NR¹²R¹³, —NR¹²R¹³, or trialkylsilyl substituents,

R¹² and R¹³ in each instance, are independently H or unsubstituted or substituted C₁₋₁₀alkyl with one or more aryl, heteroalkyl, heterocyclyl, or hetaryl substituents, wherein each of said alkyl, aryl, heteroalkyl, heterocyclyl, or hetaryl groups is unsubstituted or substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl) substituents.

In some embodiments X is CH; Z is O; L is —O—; R¹, R², and R³, each R⁹ is independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R¹¹ substituents; and R⁴, R⁵, R⁶, R⁷ and R⁸ are each independently selected from the group of hydrogen, —OH, —R¹², —OR¹², NH₂, and —NR¹²R¹³. In some embodiments at least three of R⁴, R⁵, R⁶, R⁷ and R⁸ are hydrogen. In some embodiments R⁴, R⁷ and R⁸ are hydrogen, and R⁵ and R⁶ are each independently selected from the group of hydrogen, —OH and —OR¹². In some embodiments R³ is —OH, —OR¹², or —O-carbohydrate. In other embodiments R³ is —OH or —O-carbohydrate. In some embodiments the carbohydrate is a monosaccharide, a disaccharide, or a polysaccharide. In other embodiments the monosaccharide is selected from the group consisting of glucose, galactose, and fructose. In additional embodiments the disaccharide is selected from the group consisting of sucrose, lactose, maltose and rutinose. In further embodiments, the polysaccharide is selected from the group consisting of starch, glycogen, and cellulose.

In one aspect of the invention, a compound of Formula III, its pharmaceutically acceptable salt, ester, prodrug, stereoisomer or tautomer is provided:

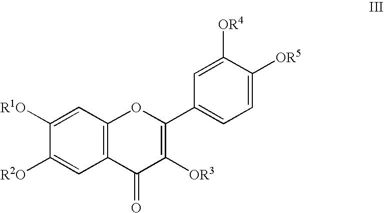

wherein R¹, R², R³, R⁴, and R⁵ are each independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R⁶ substituents,

R⁶ is halogen, —OR⁷, —SH, NH₂, —NR⁷R⁸, —CO₂R⁷, —CO₂aryl, —C(═O)NR⁷R⁸, —NO₂, —CN, —S(O)₀₋₂C₁₋₁₀alkyl, —S(O)₀₋₂aryl, —SO₂NR⁷R⁸, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR⁷R⁸, —SO₂ NR⁷R⁸, or —NR⁷R⁸ substituents,

R⁷ and R⁸ are independently H or unsubstituted or substituted C₁₋₁₀alkyl with one or more aryl, heteroalkyl, heterocyclyl, or hetaryl substituents, wherein each of said alkyl, aryl, heteroalkyl, heterocyclyl, or hetaryl groups is unsubstituted or substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂aryl, —SO₂N(aryl), —SO₂N(C₁₋₁₀alkyl)(C₁₋₁₀alkyl), or —SO₂NH(C₁₋₁₀alkyl) or substituents.

In some embodiments R³ is hydrogen or carbohydrate. In other embodiments, R⁴ and R⁵ are each independently hydrogen, C₁₋₁₀alkyl, or carbohydrate, each of which except for hydrogen is unsubstituted or substituted by one or more independent R⁶ substituents. In some embodiments R¹ and R² are each independently hydrogen, C₁₋₁₀alkyl, or carbohydrate, each of which except for hydrogen is unsubstituted or substituted by one or more independent R⁶ substituents. In other embodiments R¹, R², R⁴, and R⁵ are each independently hydrogen, unsubstituted C₁₋₁₀alkyl, or carbohydrate. In some embodiments R¹, R², R⁴, and R⁵ are each independently hydrogen or unsubstituted C₁₋₁₀alkyl, and R³ is hydrogen or carbohydrate. In some embodiments the carbohydrate is a monosaccharide, a disaccharide, or a polysaccharide. In other embodiments, the monosaccharide is selected from the group consisting of glucose, galactose, and fructose. In some embodiments, the disaccharide is selected from the group consisting of sucrose, lactose, maltose and rutinose. In other embodiments, the polysaccharide is selected from the group consisting of starch, glycogen, and cellulose.

In some embodiments, R¹, R², R⁴, and R⁵ are each independently hydrogen, C₁₋₆alkyl, C₂₋₆alkenyl, C₂₋₆alkynyl, aryl, heteroaryl, heterocyclyl, C₃₋₇cycloalkyl, or carbohydrate; and R³ is hydrogen or carbohydrate.

In some embodiments, R¹, R² are each hydrogen; R⁴, and R⁵ are each independently hydrogen, C₁₋₆alkyl, C₂₋₆alkenyl, C₂₋₆alkynyl, aryl, heteroaryl, heterocyclyl, C₃₋₇cycloalkyl, or carbohydrate; and R³ is hydrogen or carbohydrate.

In some embodiments, R¹, R², R⁴, and R⁵ are each hydrogen, and R³ is hydrogen or carbohydrate. In other embodiments, R³ is hydrogen or carbohydrate. In some embodiments, R³ is carbohydrate. In some embodiments R³ is rutinose. In one embodiment, the compound of Formula III is the following structure:

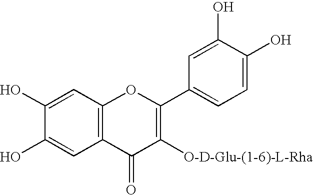

In another embodiment, the compound of Formula III is selected from the group consisting of compound 6, 9, 10, 20, and 21.

The invention also embraces isolated compounds. An isolated compound refers to a compound which represents at least 0.1%, 1%, 10%, 20%, 50%, 80%, 90%, 95%, or 99% of the compound present in a mixture.

In another aspect of the invention, a pharmaceutical composition is provided comprising the compound of Formula I, II, or III, its pharmaceutically acceptable salt, ester, prodrug, stereisomer or tautomer, and a pharmaceutically acceptable carrier or excipient. Preferably, the amount of the compound of Formula I, II, or III, its pharmaceutically acceptable salt, ester, prodrug, stereisomer or tautomer in the pharmaceutical composition is at least 0.1%, 0.5%, 1%, 5%, 10%, 20%, 50%, or 80% based on the total weight of the composition.

The pharmaceutically acceptable carrier or excipient may include a dilutent, stabilizers (to promote long term storage), adjuvants, vehicles, emulsifiers, binding agents, thickening agents, suitable additives including agents to promote the cellular uptake of the active agents in the composition such as liposomes, salts, preservatives, and the like, depending on the route of administration. In addition, the compound of Formula I, II, or III, may be incorporated into sustained-release preparations and formulations.

The pharmaceutical composition may be in a dosage formulation suitable for administration orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, transurethrally, topically, or via an implanted reservoir.

The term parenteral used herein is intended to include subcutaneous, intravenous, intracoronary, and intramuscular injection. The exact formulation, route of administration, and dosage of active compound can be chosen by the individual physician in view of the subject's condition. The compound of Formula I, II, or III, is administered in an effective amount to achieve its intended purpose and it will be dependent upon the type of disease to be treated, the severity and course of the disease, whether the compound is administered for preventative or therapeutic purposes, previous therapy, the subject being treated, the subject's weight, the manner of administration and the judgment of the prescribing physician. The compound of Formula I, II, or III, is suitable to be administered to the subject at one time or over a series of treatments, for example at a dose of 0.01-3000 mg/Kg, 0.1-1000 mg/Kg, 0.5-500 mg/Kg, 1-200 mg/Kg, 1-100 mg/Kg, or 1-50 mg/Kg.

In another aspect of the invention, a physiological composition is provided comprising the compound of Formula I, II, or III, its physiologically acceptable salt, ester, prodrug, stereisomer or tautomer, and a physiologically acceptable carrier or excipient. Preferably, the amount of the compound of Formula I, II, or III, its physiologically acceptable salt, ester, prodrug, stereisomer or tautomer in the physiological composition is at least 0.1%, 0.5%, 1%, 5%, 10%, 20%, 50%, or 80% based on the total weight of the composition.

In yet another aspect of the invention, a method of treating, preventing or reducing the risk of developing a disease associated with abnormal cell proliferation in a mammal including a human, comprising administering to a subject in need thereof an effective amount of a pharmaceutical composition comprising a compound of Formula I, II, or III.

In yet another aspect of the invention, a method of treating, preventing or reducing the risk of developing a disease associated with abnormal angiogenesis in a mammal including a human, comprising administering to a subject in need thereof an effective amount of a pharmaceutical composition comprising a compound of Formula I, II, or III.

In another aspect of the invention, a unit dosage form comprising a pharmaceutical composition comprising the compound of Formula I, II, or III, its pharmaceutically acceptable salt, ester, prodrug, stereisomer or tautomer within the range from about 1 mg to about 10 g; and a pharmaceutically acceptable carrier, diluent or excipient. In one embodiment, the range of the amount of a compound of Formula I, II, or III, or a pharmaceutically acceptable salt, ester, prodrug, stereoisomer or tautomer in the unit dosage form is from about 10 mg to about 1000 mg. In another embodiment, the range of the amount of a compound of Formula I, II, or III, or a pharmaceutically acceptable salt, ester, prodrug, stereoisomer or tautomer in the unit dosage form is from about 10 mg to about 500 mg. In a further embodiment, the range of the amount of a compound of Formula I, II, or III, or a pharmaceutically acceptable salt, ester, prodrug, stereoisomer or tautomer in the unit dosage form is from about 10 mg to about 100 mg.

In yet another aspect of the present invention, a kit for treating or preventing a disease or condition, comprising: a container or vessel comprising a pharmaceutical composition comprising the compound of Formula I, II, or III, its pharmaceutically acceptable salt, ester, prodrug, stereisomer or tautomer. The kit may further include written instructions for using said pharmaceutical composition for treating or preventing said disease or condition.

Preferable indications that may be treated using the compounds or compositions of the present invention include those involving undesirable or uncontrolled cell proliferations and/or angiogenesis. Such indications include benign tumors, various types of cancers such as primary tumors and tumor metastasis, hematologic disorders (e.g., leukemia, myelodysplastic syndrome and sickle cell anemia), restenosis (e.g., coronary, carotid, and cerebral lesions), abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types of benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor, cells become undifferentiated, do not respond to the body's growth control signals, and multiply in an uncontrolled manner. The malignant tumor is invasive and capable of spreading locally and to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include leukemia, breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalceria, renal cell tumor, polycythermia vera, adenocarcinoma, glioblastoma multiforma, leukemias, lymphomas, malignant melanomas, epidermoid carcinomas, and other carcinomas and sarcomas.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

Treatment of abnormal cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome.

The proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

Abnormal angiogenesis that may be treated using this invention include those abnormal angiogenesis accompanying rheumatoid arthritis, ischemic-reperfusion related brain edema and injury, cortical ischemia, ovarian hyperplasia and hypervascularity, (polycystic ovary syndrome), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Diseases associated with abnormal angiogenesis require or induce vascular growth. For example, corneal angiogenesis involves three phases: a pre-vascular latent period, active neovascularization, and vascular maturation and regression. The identity and mechanism of various angiogenic factors, including elements of the inflammatory response, such as leukocytes, platelets, cytokines, and eicosanoids, or unidentified plasma constituents have yet to be revealed.

The particular dosage of these agents required to inhibit angiogenesis and/or angiogenic diseases may depend on the severity of the condition, the route of administration, and related factors that can be decided by the attending physician. Generally, accepted and effective daily doses are the amount sufficient to effectively inhibit angiogenesis and/or angiogenic diseases.

According to this embodiment, the composition of the present invention may be used to treat a variety of diseases associated with undesirable angiogenesis such as retinal/choroidal neuvascularization and corneal neovascularization. Examples of retinal/choroidal neuvascularization include, but are not limited to, Best's disease, myopia, optic pits, Stargart's disease, Paget's disease, vein occlusion, artery occlusion, sickle cell anemia, sarcoid, syphilis, pseudoxanthoma elasticum carotid abostructive diseases, chronic uveitis/vitritis, mycobacterial infections, Lyme's disease, systemic lupus erythematosis, retinopathy of prematurity, Eale's disease, diabetic retinopathy, macular degeneration, Bechet's diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of corneal neuvascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

In yet another embodiment of the present invention, a method is provided for treating chronic inflammatory diseases associated with abnormal angiogenesis. The method comprises administering to a patient suffering from a chronic inflammatory disease associated with abnormal angiogenesis a pharmaceutical composition of the present invention. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis using the composition of the present invention may prevent the formation of the granulomas, thereby alleviating the disease. Examples of chronic inflammatory disease include, but are not limited to, inflammatory bowel diseases such as Crohn's disease and ulcerative colitis, psoriasis, sarcoidois, and rhematoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea. These inflammatory bowel diseases are generally caused by chronic granulomatous inflammation throughout the gastrointestinal tract, involving new capillary sprouts surrounded by a cylinder of inflammatory cells. Inhibition of angiogenesis by the composition of the present invention should inhibit the formation of the sprouts and prevent the formation of granulomas. The inflammatory bowel diseases also exhibit extra intestinal manifestations, such as skin lesions. Such lesions are characterized by inflammation and angiogenesis and can occur at many sites other the gastrointestinal tract. Inhibition of angiogenesis by the composition of the present invention should reduce the influx of inflammatory cells and prevent the lesion formation.

Sarcoidois, another chronic inflammatory disease, is characterized as a multisystem granulomatous disorder. The granulomas of this disease can form anywhere in the body and, thus, the symptoms depend on the site of the granulomas and whether the disease is active. The granulomas are created by the angiogenic capillary sprouts providing a constant supply of inflammatory cells. By using the composition of the present invention to inhibit angionesis, such granulomas formation can be inhibited. Psoriasis, also a chronic and recurrent inflammatory disease, is characterized by papules and plaques of various sizes. Treatment using the composition of the present invention should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using the composition of the present invention alone or in conjunction with other anti-RA agents should prevent the formation of new blood vessels necessary to maintain the chronic inflammation and provide the RA patient relief from the symptoms.

A wide variety of anti-neoplastic agents may be used in conjunction with the compound of Formula I, II, or III, its pharmaceutically acceptable salt, ester, prodrug, stereisomer or tautomer, for treating various diseases such those described above.

The antineoplastic agent may be an antibiotic agent. Antibiotic agents are a group of anticancer drugs that are produced in a manner similar to antibiotics by a modification of natural products. Examples of antibiotic agents include, but are not limited to, anthracyclines (e.g., doxorubicin, daunorubicin, epirubicin, idarubicin and anthracenedione), mitomycin C, bleomycin, dactinomycin, plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and form an activated complex, which then binds to bases of DNA, causing strand scissions and cell death. Such a combination therapy may have therapeutic synergistic effects on cancer and reduce sides affects associated with these chemotherapeutic agents.

The antineoplastic agent may be an antimetabolic agent. Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), decitabine, 5-azacytidine, methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Such a combination therapy may have therapeutic synergistic effects on cancer and reduce sides affects associated with these chemotherapeutic agents.

The antineoplastic agent may also be a plant-derived agent. Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), water soluble or insoluble camptothecin (e.g., 20(S)-camptothecin, 9-nitro-camptothecin, 9-nitro-camptothecin, and topotecan), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Camptothecin is believed to be a potent inhibitor of the nuclear enzyme DNA topoisomerase I (topo-I), which is responsible for “relaxation” of supercoiled double-stranded DNA by creating single-stranded breaks through which another DNA strand can pass during transcription. Topo-I reseals the break allowing DNA replication to occur. Inhibition of topo-I leads to the formation of stable DNA-topoisomerase complexes, with eventual formation of irreversible double-stranded DNA breaks, leading to apoptosis and/or other forms of cell death. Podophyllotoxins such as etoposide are believed to interfere with DNA synthesis by interacting with topoisomerase II, leading to DNA strand scission. Such a combination therapy may have therapeutic synergistic effects on cancer and reduce side affects.

The antineoplastic agent may be a biologic agent. Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Such a combination therapy may have therapeutic synergistic effects on cancer, enhance the patient's immune responses to tumorigenic signals, and reduce potential sides affects associated with the biologic agent.

Cytokines possess profound immunomodulatory activity. Some cytokines such as interleukin-2 (IL-2, aldesleukin) and interferon-α (IFN-α) demonstrate antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and non-self, Examples of interleukins that may be used in conjunction with a DNA methylation inhibitor include, but are not limited to, interleukin 2 (IL-2), and interleukin 4 (IL-4), interleukin 12 (IL-12).

Interferon-α includes more than 23 related subtypes with overlapping activities, all of the IFN-α subtypes within the scope of the present invention. IFN-α has demonstrated activity against many solid and hematologic malignancies, the later appearing to be particularly sensitive. Examples of interferons include, but are not limited to, interferon-α, interferon-β (fibroblast interferon) and interferon-γ (fibroblast interferon).

Other cytokines that may be used in conjunction with the compound or composition of the present invention include those cytokines that exert profound effects on hematopoiesis and immune functions. Examples of such cytokines include, but are not limited to erythropoietin (epoietin-α), granulocyte-CSF (filgrastin), and granulocyte, macrophage-CSF (sargramostim).

Immuno-modulating agents other than cytokines may also be used in conjunction with the compound or composition of the present invention to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors overexpress the HER2 protein. Combination therapy including the compound or composition of the present invention and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and malignant CD20⁺ pre-B and mature B cells. RITUXAN® is used as single agent for the treatment of patients with relapsed or refractory low-grade or follicular, CD20⁺ B cell non-Hodgkin's lymphoma. Combination therapy including the compound or composition of the present invention and RITUXAN® may have therapeutic synergistic effects not only on lymphoma, but also on other forms or types of malignant tumors.

Tumor suppressor genes are genes that express proteins that control entry or progression through the cell cycle. Some tumor suppressor proteins also promote apoptosis. Mutations in tumor suppressor genes cause the cell to ignore one or more of the components of the network of inhibitory signals controlling the cell cycle or to ignore commands to undergo apoptosis, resulting in uncontrolled proliferation. The activation of expression of tumor suppressor genes though the administration of pharmaceutical agents or by gene therapy may provide a means to control or inhibit the uncontrolled cell proliferation. Examples of the tumor suppressor genes that can be used include, but are not limited to, DPC-4, NF-1, NF-2, RB, p53, WT1, BRCA1 and BRCA2.

Functional Assays

Compounds of Formula I, II, or III can be assayed in vitro and/or in vivo for biological activity. Candidate compounds are identified by the inhibition or suppression of VEGF, VEGF R1, VEGF R2, CDK1, CDK2, Bcl-2, and/or p21 expression. Candidate compounds can be further identified by a reduction of cell proliferation, an increase in apoptosis, and/or a reduction in the formation of neovasculature. A compound with the desired potency and selectivity can serve as a compound for the compositions and methods of treatment of the present invention. Alternatively, active compounds can be used as lead compounds for further rounds of drug design, synthesis and testing.

Inhibition of Angiogenesis Regulator VEGF Expression

Following exposure to 0.001 to 1,000 μM of a test compound, in culture for 0.1-168 hours, VEGF expressing cell lines are examined for their expression of VEGF. Suitable cell lines include DU-145 and LNCaP. An inhibitor effect is detected when a decrease in VEGF expression is found to be at least 10%, more preferably at least 20%, 30%, 40%, 50%, 60%, 70%, 80%, 90%, or 95% compared to a untreated cells.

VEGF expression can be measured by any standard way known in the art including flow cytometry using labeled anti-VEGF antibodies; immunohistochemical examination of slides containing cells using anti-VEGF antibodies that are conjugated to fluorescent labels or enzymes; or immunoblotting of cell lysates. Additionally, mRNA expression of the VEGF gene can be determined by performing nucleic acid amplification, such as with PCR.

Similar measurements of VEGF expression can be performed on tissue samples from animals or patients, include tumor samples.

Inhibition of VEGF Receptor

Following exposure to 0.001 to 1,000 μM of a test compound, in culture for 0.1-168 hours, cell lines are examined for their expression of VEGF Receptor I (VEGF R1) and VEGF Receptor II (VEGF R2). An inhibitor effect is detected when a decrease in VEGF R1 or VEGF R2 expression is found to be at least 10%, more preferably at least 20%, 30%, 40%, 50%, 60%, 70%, 80%, 90%, or 95% compared to untreated cells.

VEGF R1 and VEGF R2 expression can be measured by any standard way known in the art including flow cytometry using labeled anti-VEGF R1 or VEGF R2 antibodies; immunohistochemical examination of slides containing cells using anti-VEGF R1 or VEGF R2 antibodies that are conjugated to fluorescent labels or enzymes; or immunoblotting of cell lysates with VEGF R1 or VEGF R2. Additionally, mRNA expression of the VEGF receptor genes can be determined by performing nucleic acid amplification, such as with PCR.

Similar measurements of VEGF receptor expression can be performed on tissue samples, from animals or patients, include tumor samples.

Inhibition of Cell Proliferation

Following exposure to 0.001 to 1,000 μM of a test compound, in culture for 0.1-168 hours, cell lines are examined for their proliferation/survival status using methods such as direct cell number counting, BrdU or H³-thymidine incorporation, tetrazolium salt 3, [4,5-dimethylthiazol-2-yl]-2,5-diphenyltetrazolium bromide (MTT) cell proliferation assay, or 3-(4,5-dimethylthiazol-2-yl)-5-(3-carboxymethoxyphenyl)-2-(4-sulfophenyl)-2H-tetrazolium (MTS) cell proliferation assay. An inhibitory effect is detected when a decrease in cell proliferation is found to be at least 10%, more preferably at least 20%, 30%, 40%, 50%, 60%, 70%, 80%, 90%, or 95% compared to untreated cells.

Both a concentration range of a test compound and the effect of the length of exposure to the test compound can be examined with the above assays.

Cell Cycle Arrest

Following exposure to 0.001 to 1,000 μM of a test compound, in culture for 0.1-168 hours, cell lines are examined using flow cytometry to determine the distribution of the cells in the different phases of the cell cycle. Total cellular DNA content can be measured with propidium iodide and the amount of BrdUrd incorporated into cellular DNA during sample period can be measured by a fluorescently labeled monoclonal antibody against BrdUrd. Through the use of bivariate DNA/BrdUrd distribution analysis, G1- and G2M-phase cells can be identified by their low BrdUrd-linked fluorescence and S-phase cells identified by their high BrdUrd-linked fluorescence. An inhibitory effect on progression through the cell cycle is detected when an increase in the number of cells found in G1 phase is found to be at least 10%, more preferably at least 20%, 30%, 40%, 50%, 60%, 70%, 80%, 90%, or 95% compared to untreated cells.

Similar measurements of the percentage of cells in the different phases of the cell cycle can be performed on tissue samples, include tumor samples.

Inhibition of CDK1 and CDK2 Expression

Following exposure to 0.001 to 1,000 μM of a test compound, in culture for 0.1-168 hours, the inhibition of CDK1 and/or CDK2 expression in a cell line can be ascertained by performing a western blot (immunoblot). Detection of the bound anti-CDK1 and/or anti-CDK2 antibody can be by any of the standard means including colorimetric, chemiluminescent, radioactive or fluorescent means. An inhibitory effect on the expression of CDK1 and/or CDK2 is detected when a decrease in CDK1 and/or CDK2 expression is found to be at least 10%, more preferably at least 20%, 30%, 40%, 50%, 60%, 70%, 80%, 90%, or 95% compared to untreated cells. Additionally, mRNA expression of the CDK1 and/or CDK2 gene can be determined by performing nucleic acid amplification, such as with PCR.

Similar measurements to ascertain the expression level of CDK1 and/or CDK2 can be performed on tissue samples, include tumor samples.

Inhibition of Bcl-2 Expression

Following exposure to 0.001 to 1,000 μM of a test compound, in culture for 0.1-168 hours, the inhibition of Bcl-2 expression in a cell line can be ascertained by performing a western blot (immunoblot). Detection of bound anti-Bcl-2 antibody can be by any of the standard means including colorimetric, chemiluminescent, radioactive or fluorescent means. An inhibitory effect on the expression of Bcl-2 is determined when a decrease in Bcl-2 expression is found to be at least 10%, more preferably at least 20%, 30%, 40%, 50%, 60%, 70%, 80%, 90%, or 95% compared to untreated cells. Additionally, mRNA expression of the Bcl-2 gene can be determined by performing nucleic acid amplification, such as with PCR.

Similar measurements to ascertain the expression level of Bcl-2 can be performed on tissue samples, include tumor samples.

Inhibition of p21 Expression

Following exposure to 0.001 to 1,000 μM of a test compound, in culture for 0.1-168 hours, the inhibition of p21 expression in a cell line can be ascertained by performing a western blot (immunoblot). Detection of bound anti-p21 antibody can be by any of the standard means including colorimetric, chemiluminescent, radioactive or fluorescent means. An inhibitory effect on the expression of p21 is determined when a decrease in p21 expression is found to be at least 10%, more preferably at least 20%, 30%, 40%, 50%, 60%, 70%, 80%, 90%, or 95% compared to untreated cells. Additionally, mRNA expression of the p21 gene can be determined by performing nucleic acid amplification, such as with PCR.

Similar measurements to ascertain the expression level of p21 can be performed on tissue samples, include tumor samples.

Promotion of Apoptosis

Following exposure to 0.001 to 1,000 μM of a test compound, in culture for 0.1-168 hours, the induction of apoptosis in a cell line can be ascertained by various known methods in the art. For example, apoptosis can be measured by flow cytometry as part of the annexin V, terminal transferase-mediated biotinylated 16-desoxy-uridene triphosphate nick-end labeling (TUNEL), or Apo2.7 (7A6 antigen) assays. An increase in apoptosis of at least 10%, 20%, 30%, 40%, 50%, 60%, 70%, 80%, 90%, or 95% compared to untreated cells is recognized as a positive effect on inducing apoptosis.

Inhibition of Tumor Growth in Xenograph Animal Model

Following exposure to 0.01 to 1,000 mg/kg body weight of a test compound for 1-100 days, inhibition of tumor growth in a xenograph animal model can be ascertained by various known methods in the art. For example, tumor size can be measured periodically (such as once every three days) from the time of tumor implantation to 30 days after tumor implantation. An inhibitory effect on tumor growth is determined when a decrease in tumor size is found to be at least 10%, 20%, 30%, 40%, 50%, 60%, 70%, 80%, 90%, or 95% compared to the tumors of untreated animals.

Pharmaceutical Compositions and Administration

The present invention also provides pharmaceutical compositions comprising an effective amount of a compound of Formula I, II, or III, for inhibiting abnormal cell proliferation and angiogenesis in both prophylactic and therapeutic applications. Pharmaceutical compositions of the invention are suitable for use in a variety of drug delivery systems. Suitable formulations for use in the present invention are found in Remnington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., oral, subcutaneous, transdermal, intramuscular, intravenous, or intraperitoneal. Preferred routes of administering the pharmaceutical compositions include systemic or local delivery to an organ or tissue suffering from a condition caused by abnormal cell proliferation or angiogenesis at daily doses of about 0.01-5000 mg, preferably 5-500 mg, of a compound of Formula I, II, or III, for a 70 kg adult human per day. The appropriate dose may be administered in a single daily dose or as divided doses presented at appropriate intervals, for example as two, three, four, or more subdoses per day.

For preparing pharmaceutical compositions containing a compound of Formula I, II, or III, inert and pharmaceutically acceptable carriers are used. The pharmaceutical carrier can be either solid or liquid. Solid form preparations include, for example, powders, tablets (e.g., compressed tablets, sugar-coated tablets, film-coated tablets, and enteric coated tablets), dispersible granules, capsules (e.g., hard or soft gelatin or non-gelatin capsules), cachets, and suppositories. A solid carrier can be one or more substances that can also act as diluents (e.g., lactose and other sugars, starch, dicalcium phosphate and cellulosic materials), flavoring agents, solubilizers, lubricants, suspending agents, binders (e.g., hydroxypropylmethylcellulose, polyvinyl pyrilodone, other cellulosic materials and starch), or tablet disintegrating agents (e.g., starch polymers and cellulosic materials) and lubricating agents (e.g., stearates and talc). Solid dosage forms can be coated using coatings and techniques well known in the art.

In powders, the carrier is generally a finely divided solid that is in a mixture with the finely divided active component, e.g., a compound of Formula I, II, or III. In tablets, the active ingredient (a compound of Formula I, II, or III) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of a compound of Formula I, II, or III. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of a compound of Formula I, II, or III, with encapsulating material as a carrier providing a capsule in which a compound of Formula I, II, or III (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g a compound of Formula I, II, or III) or sterile solutions of the active component in solvents comprising water, buffered water, saline, PBS, excipients such as solubility-altering agents (e.g., ethanol, propylene glycol and sucrose) and polymers (e.g., polycaprylactones and PLGA's) are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a compound of Formula I, II, or III) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing a compound of Formula I, II, or III, can be administered for prophylactic and/or therapeutic treatments. In therapeutic applications, compositions are administered to a patient already suffering from a condition that may be exacerbated by angiogenesis and/or uncontrolled cell proliferation supporting tumor growth, in an amount sufficient to prevent, cure, reverse, or at least partially slow or arrest the symptoms of the condition and its complications. An amount adequate to accomplish this is defined as a “therapeutically effective dose.” Amounts effective for this use will depend on the severity of the disease or condition and the weight and general state of the patient, but generally range from about 0.1 mg to about 5,000 mg of a compound of Formula I, II, or III, per day for a 70 kg patient, with dosages of from about 5 mg to about 500 mg of the compound per day for a 70 kg patient being more commonly used.

In prophylactic applications, pharmaceutical compositions containing a compound of Formula I, II, or III, are administered to a patient susceptible to or otherwise at risk of developing a disease or condition characterized by angiogenesis and/or uncontrolled cell proliferation. The administered amount is sufficient to delay or prevent the onset of the symptoms. Such an amount is defined to be a “prophylactically effective dose.” In this use, the precise amounts of a compound of Formula I, II, or III, again depend on the patient's state of health and weight, but generally range from about 0.1 mg to about 5,000 mg of the compound for a 70 kg patient per day, more commonly from about 5 mg to about 500 mg for a 70 kg patient per day.

Single or multiple administrations of the compositions can be carried out with dose levels and pattern being selected by the treating physician. In any event, the pharmaceutical formulations should provide a quantity of a compound of Formula I, II, or III, sufficient to effectively inhibit angiogenesis and/or cell proliferation in the patient, either therapeutically or prophylatically.

Pharmaceutical Formulations

When used for pharmaceutical purposes, a compound of Formula I, II, or III is generally formulated in a suitable buffer, which can be any pharmaceutically acceptable buffer, such as phosphate buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize a compound of Formula I, II, or III. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

Oral delivery systems include solid dosage forms such as tablets (e.g., compressed tablets, sugar-coated tablets, film-coated tablets, and enteric coated tablets), capsules (e.g., hard or soft gelatin or non-gelatin capsules), blisters, and cachets. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). The solid dosage forms can be coated using coatings and techniques well known in the art.

Oral liquid dosage forms include solutions, syrups, suspensions, emulsions, elixirs (e.g., hydroalcoholic solutions), and powders for reconstitutable delivery systems. The formulations can contain one or more carriers or excipients, such as suspending agents (e.g., gums, zanthans, cellulosics and sugars), humectants (e.g., sorbitol), solubilizers (e.g., ethanol, water, PEG, glycerin, and propylene glycol), surfactants (e.g., sodium lauryl sulfate, Spans, Tweens, and cetyl pyridine), emulsifiers, preservatives and antioxidants (e.g., parabens, vitamins E and C, and ascorbic acid), anti-caking agents, coating agents, chelating agents (e.g., EDTA), flavorants, colorants, and combinations thereof. The compositions can be formulated as a food or beverage (e.g., a shake) containing buffer salts, flavoring agents, coloring agents, sweetening agents, and combinations thereof.

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Exemplary permeation enhancing compositions, polymer matrices, and mucoadhesive gel preparations for transdermal delivery are disclosed in U.S. Pat. No. 5,346,701.

Administration of Formulations

The formulations containing a compound of Formula I, II, or III, of the invention can be delivered to any tissue or organ using any delivery method known to the ordinarily skilled artisan. Effective dosage of the formulations will vary depending on many different factors, including means of administration, target site, physiological state of the patient, and other medicines administered. Thus, treatment dosages will need to be titrated to optimize safety and efficacy. In determining the effective amount of a compound of Formula I, II, or III to be administered, the physician should evaluate the particular compound used, the disease state being diagnosed; the age, weight, and overall condition of the patient, circulating plasma levels, compound toxicities, and progression of the disease. The size of the dose also will be determined by the existence, nature, and extent of any adverse side-effects that accompany the administration of a particular compound. To practice the present invention, doses ranging from about 10 ng-50 g, 100 ng-10 g, 1 μg-1 g, or 10-1,000 mg of a compound of Formula I, II, or III per patient per day are typical. Doses generally range between about 0.01)_{g} and about 1 g per kilogram of body weight, preferably between about 0.11 g and about 500 mg/kg of body weight, more preferably between about 1 μg and about 50 mg/kg of body weight, and most preferably between about 10 μg and about 5 mg/kg of body weight.

Kits

The invention also provides kits for inhibiting angiogenesis and/or cell proliferation according to the method of the present invention. The kits typically include a container that contains a pharmaceutical composition having an effective amount of a compound of Formula I, II, or III, as well as informational material containing instructions on how to dispense the pharmaceutical composition, including description of the type of patients who may be treated (e.g., a person at risk of developing advanced tumor mass), the schedule (e.g., dose and frequency), route of administration, and the like.

### EXAMPLES

The following examples are provided by way of illustration only and not by way of limitation. Those of skill in the art will readily recognize a variety of non-critical parameters that could be changed or modified to yield essentially similar results

1. Synthesis and Preparation of Compounds of the Present Invention

A compound of Formula I, II, or III can be prepared by any suitable method known in the art, or by the following processes which form part of the present invention. The compounds of general structure of Formula I, II, or III can be prepared by methods shown in examples below.

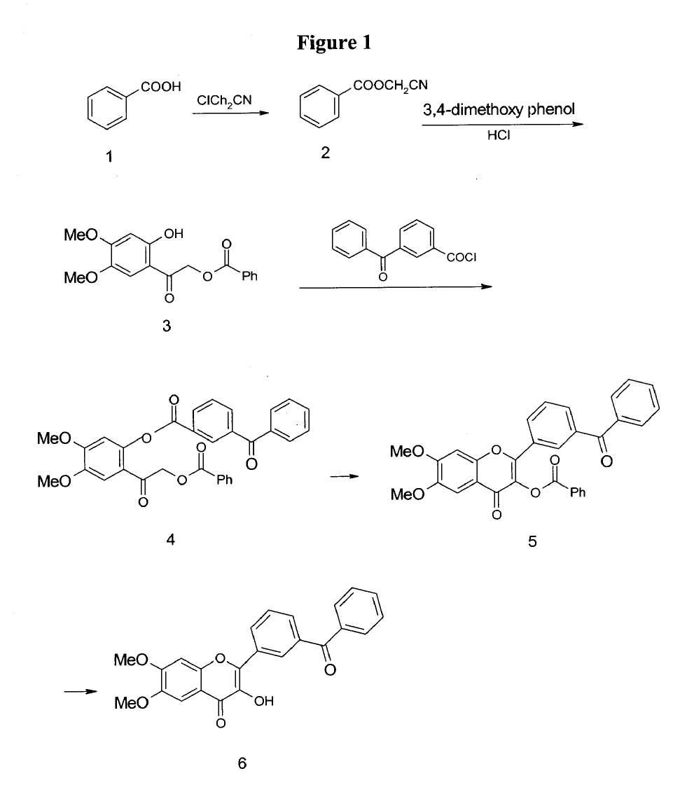

In the synthesis of compounds of Formula I, II, or III, protecting compounds and protecting groups, like benzyl chloroformate and trichloroethyl chloroformate, which are well known to persons skilled in the art, can be used. These protecting compounds and groups are removed in the final steps of the synthesis under basic, acidic, or hydrogenolytic conditions which are readily apparent to those skilled in the art. By using appropriate starting materials, and manipulation and protection of chemical functionalities, synthesis of compounds of Formula I, II, or III not specifically set forth herein can be accomplished by methods analogous to the schemes shown in FIGS. 1-3.

Compounds of Formula I, II, or III can be converted to other compounds of Formula I, II, or III. Thus, for example, when a compound contains a substituted aromatic ring, it is possible to prepare another suitably substituted compound of Formula I, II, or III. Examples of appropriate interconversions include, but are not limited to, OR_{b} to hydroxy by suitable means (e.g., using an agent such as BBr₃, SnCl₂, or a palladium catalyst, such as palladium-on-carbon), or amino to substituted amino, such as alkylamine, using standard acylating or sulfonylating conditions.

The following illustrates specific examples of compounds of Formula I, II, or III and synthetic routes to compounds and are not meant to be limiting.

FIG. 1 illustrates the synthesis scheme of an embodiment of Formula I, II, or III.

Accordingly, 0.5 mol Benzoic acid (Compound 1) and 1 mol triethylamine were dissolved in 400 ml dry dichloromethane. 65 ml chloroacetonitrile was added to the mixture and incubated at room temperature overnight. The triethylamine salt was filtered out under vacuum and the remaining solution was washed twice with 5% Na₂CO₃, 2 N HCl, and H₂O, respectively. The organic layer was separated and dried with MgSO₄. The solvent was removed by vacuum evaporation. The resulting light yellow solution contains benzoxy acetonitrile (Compound 2).

24.39 g benzoxy acetonitrile (Compound 2) and 0.15 mol 3,4-dimethoxy phenol were added to 160 ml dry ether cooled in an ice water bath. 8.0 g dry ZnCl₂ was added to the mixture. The reaction mixture was then stirred while adding HCl gas for 2 hr. After yellowish green precipitates were formed, the container was sealed and frozen for 48 hr. Afterwards the ether was discarded from the container and 600 ml 50% ethanol was added to the mixture. The reaction mixture was then refluxed for 10 hr. Afterwards 100 ml ethanol was distilled from the reaction mixture under vacuum. The mixture was then cooled until yellow precipitates were formed (Compound 3).

3.16 g Compound 3 and 11.5 mmol 3-BenzoylBenzoyl chloride were dissolved in 50 ml dry pyridine and refluxed for 2 hr. The reaction mixture was then poured into a solution consisting of 100 ml ice water and 10 ml HCl. The reaction mixture was extracted twice with CH₂Cl₂, washed three times with aqueous phase of Na₂CO₃ and H₂O, respectively. The organic layer was separated and dried with MgSO₄. The solvent was removed by vacuum evaporation. A white solid was formed by re-crystallization with ethanol (Compound 4).

100 ml dry THF containing 600 mg sodium hydride (washed three times with petroleum ether before use) was added to 2.62 g Compound 4 in 100 ml dry THF. The reaction mixture was refluxed for 4 hr at room temperature. The reaction mixture was poured into a solution consisting of 600 ml ice water and 10 ml HCl, extracted twice with ether and washed three times with H₂O. The organic layer was separated and dried with MgSO₄. The solvent was removed by vacuum evaporation. A yellow solid was obtained after purification by silica gel electrophoresis using an elution buffer of 9% acetone in petroleum ether (Compound 5).

1.7 g KOH was dissolved in 50 ml MeOH and added to 7.59 g Compound 5. The reaction mixture was refluxed for 2 hr. The reaction mixture was then poured into 500 ml H₂O and H₂SO₄ was added to adjust pH to pH 2. A yellow solid was formed (Compound 6).

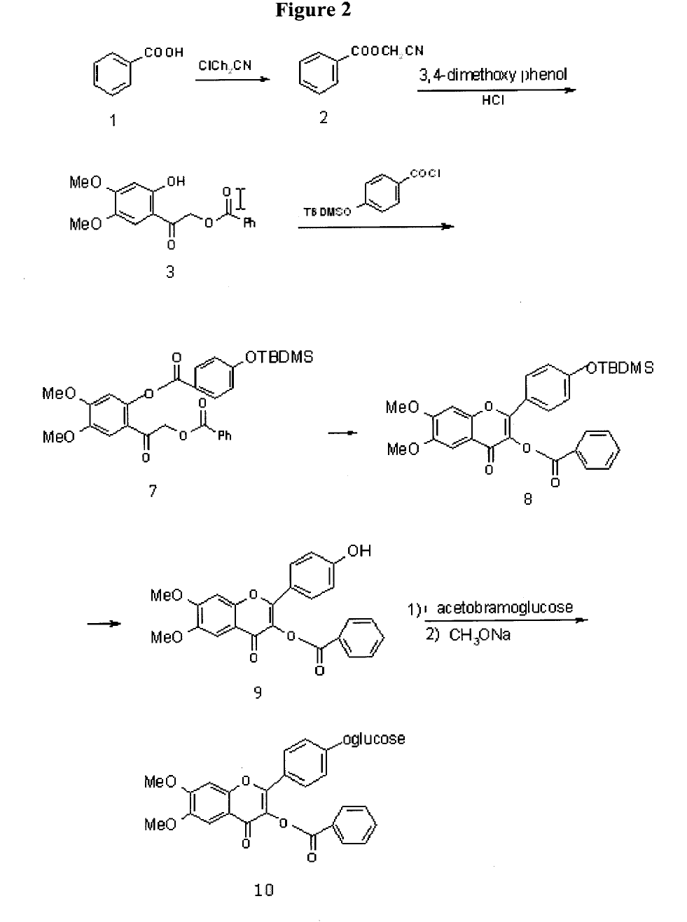

FIG. 2 illustrates the synthesis scheme of another embodiment of Formula I, II, or III.

Accordingly, 0.5 mol Benzoic acid (Compound 1) and 1 mol triethylamine were dissolved in 400 ml dry dichloromethane. 65 ml chloroacetonitrile was added to the mixture and incubated at room temperature overnight. The triethylamine salt was filtered out under vacuum and the remaining solution was washed twice with 5% Na₂CO₃, 2 N HCl, and H₂O, respectively. The organic layer was separated and dried with MgSO₄. The solvent was removed by vacuum evaporation. The resulting light yellow solution contains benzoxy acetonitrile (Compound 2).

24.39 g benzoxy acetonitrile (Compound 2) and 0.15 mol 3,4-dimethoxy phenol were added to 160 ml dry ether cooled in an ice water bath. 8.0 g dry ZnCl₂ was added to the mixture. The reaction mixture was then stirred while adding HCl gas for 2 hr. After yellowish green precipitates were formed, the container was sealed and frozen for 48 hr. Afterwards the ether was discarded from the container and 600 ml 50% ethanol was added to the mixture. The reaction mixture was then refluxed for 10 hr. Afterwards 100 ml ethanol was distilled from the reaction mixture under vacuum. The mixture was then cooled until yellow precipitates were formed (Compound 3).

3.16 g Compound 3 and 11.5 mmol 4-TBDMSOBenzoyl chloride were dissolved in 50 ml dry pyridine and refluxed for 2 hr. The reaction mixture was then poured into a solution consisting of 100 ml ice water and 10 ml HCl. The reaction mixture was extracted twice with CH₂Cl₂, washed three times with aqueous phase of Na₂CO₃ and H₂O, respectively. The organic layer was separated and dried with MgSO₄. The solvent was removed by vacuum evaporation. A white solid was formed by re-crystallization with ethanol (Compound 7).

100 ml dry THF containing 600 mg sodium hydride (washed three times with petroleum ether before use) was added to 2.75 g Compound 7 in 100 ml dry THF. The reaction mixture was refluxed for 4 hr at room temperature. The reaction mixture was poured into a solution consisting of 600 ml ice water and 10 ml HCl, extracted twice with ether and washed three times with H₂O. The organic layer was separated and dried with MgSO₄. The solvent was removed by vacuum evaporation. A yellow solid was obtained after purification by silica gel electrophoresis using an elution buffer of 9% acetone in petroleum ether (Compound 8).

5.32 g Compound 8 was dissolved in 50 ml dry THF and then added to 0.015 mol tetrabutylammonium fluoride in THF solution at 0° C. The reaction was terminated after 30 min and purified using silica gel electrophoresis to form a white solid (Compound 9).

4.18 g Compound 9 was dissolved in 50 ml dry benzene and then 3.3 g Ag2CO3 was added to the solution. After stirring the mixture at room temperature for 1 hr, 5.0 g acetobramoglucose was added and the reaction mixture was stirred at room temperature overnight. The reaction mixture was then poured into 150 ml aqueous phase of NaHCO₃. Silver salt was filtered out under vacuum. The mixture was then extracted twice with CHCl₃. The organic layer was separated and dried with MgSO₄. The solvent was removed by vacuum evaporation. Then 50 ml MeOH and MeONa were added to the mixture. The reaction mixture was stirred at room temperature overnight. Acetic acid was used to adjust pH of the solution to pH 7. The solvent was removed by vacuum evaporation. A yellow solid was obtained after purification by silica gel electrophoresis using an elution buffer of 20% acetone in petroleum ether (Compound 10).

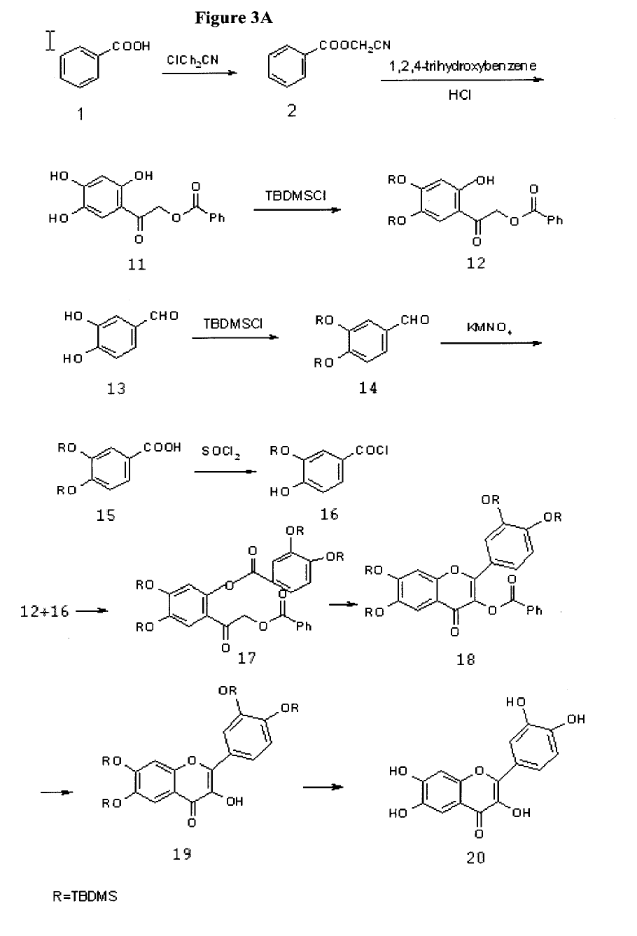

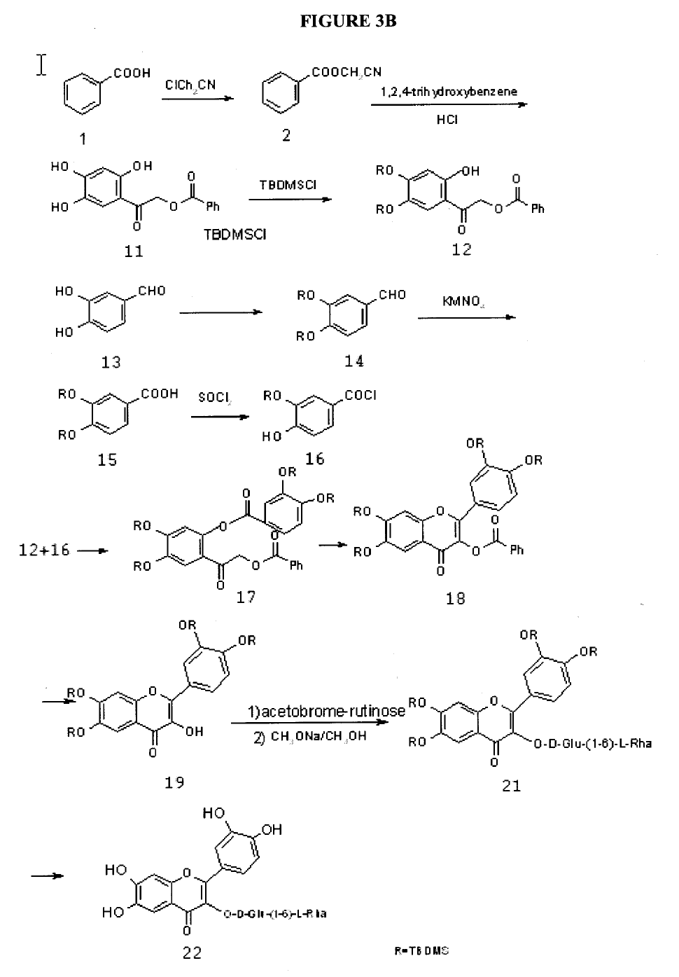

FIG. 3A illustrates the synthesis scheme of another embodiment of Formula I, II, or III.

Accordingly, 0.5 mol Benzoic acid (Compound 1) and 1 mol triethylamine were dissolved in 400 ml dry dichloromethane. 65 ml chloroacetonitrile was added to the mixture and incubated at room temperature overnight. The triethylamine salt was filtered out under vacuum and the remaining solution was washed twice with 5% Na₂CO₃, 2 N HCl, and water, respectively. The organic layer was separated and dried with MgSO₄. The solvent was removed by vacuum evaporation. The resulting light yellow solution contains benzoxy acetonitrile (Compound 2).

24.39 g benzoxy acetonitrile (Compound 2) and 0.15 mol 1,2,4-trihydroxybenzene were added to 160 ml dry ether cooled in an ice water bath. 8.0 g dry ZnCl₂ was added to the mixture. The mixture was then stirred while adding HCl gas for 2 hr. After yellowish green precipitates were formed, the container was sealed and frozen for 48 hr. Then ether was discarded from the container and 600 ml 50% ethanol was added to the mixture. The reaction mixture was refluxed for 10 hr. Afterwards 140 ml ethanol was distilled from the reaction mixture under vacuum. The mixture was then cooled until yellow precipitates were formed (Compound 11).

14.4 g Compound 11 and 0.125 mol triethylamine were dissolved in 400 ml dry dichloromethane. 0.125 mol T-butyldimethylchorosilane (TBDMSCl) was dissolved in 100 ml dry dichloromethane. The two solutions were mixed for 4 hr. The reaction mixture was then stirred overnight at room temperature. The reaction solution was washed twice with 1% HCl. The organic layer was separated and dried with MgSO₄. The solvent was removed by vacuum evaporation. After a small amount of methanol was added, white precipitates were formed in the mixture (Compound 12).

14.4 g Compound 13 and 0.125 mol triethylamine were dissolved in 400 ml dry dichloromethane. 0.125 mol T-butyldimethylchorosilane (TBDMSCl) was dissolved in 100 ml dry dichloromethane. The two solutions were mixed for 4 hr. The reaction mixture was stirred overnight at room temperature. Then the reaction solution was washed twice with 1% HCl. The organic layer was separated and dried with MgSO₄. The solvent was removed by vacuum evaporation. After a small amount of methanol was added, white precipitates were formed in the mixture (Compound 14).

14.6 g Compound 14 was added to 150 ml H₂O and heated to 70-80° C. with stirring. 0.06 mol KMnO₄ was dissolved in 180 ml H₂O and added to the Compound 14 solution. 10% KOH was added to the mixture to turn the solution alkaline. The solution was then filtered. After the filtrate was cooled, HCl was added to the filtrate to reach pH 2. Then white precipitates were formed in the filtrate (Compound 15).

3.82 g Compound 15 was dissolved in 60 ml dry THF and 0.015 mol SOCl₂ was added to the solution. The reaction mixture was stirred at room temperature overnight. Afterwards, the solvent was removed by vacuum evaporation and white precipitates were formed in the solution (Compound 16).

1 g Compound 12 and 0.9 g Compound 16 were mixed and the mixture was refluxed in 10 ml dry pyridine for 2 hr. The reaction mixture was then poured into a solution consisting of 20 ml crushed ice and 2 ml HCl. The mixture was extracted twice with CH₂Cl₂, and then washed three times with an aqueous phase of Na₂CO₃ and H₂O, respectively. The organic layer was separated and dried with MgSO₄. The solvent was removed by vacuum evaporation. The precipitates were re-crystallized with ethanol to form a white solid (Compound 17).

10 ml THF solution containing 60 mg sodium hydride (washed three times with petroleum ether before use) was added to 10 ml THF solution containing 0.44 g Compound 17. The reaction mixture was refluxed for 4 hr. The reaction mixture was then poured into a mixture consisting of 60 ml ice and 1 ml HCl. The reaction mixture was extracted twice with ether and washed three times with H₂O. The organic layer was separated and dried with MgSO4. The solvent was removed by vacuum evaporation. A yellow solid was obtained after purification by silica gel electrophoresis using an elution buffer of 9% acetone in petroleum ether (Compound 18).

0.34 g KOH was dissolved in 10 ml MeOH and then added to 2.6 g Compound 18. The reaction mixture was refluxed for 2 hr. The reaction mixture was then poured into 100 ml H₂O and H₂SO₄ was used to adjust pH to pH 2. A white solid was formed afterwards (Compound 19).

0.76 g Compound 19 was dissolved in 20 ml dry THF and added to 0.06 mol tetrabutylammonium fluoride in THF solution at 0° C. The reaction was completed after 30 min and purified using silica gel electrophoresis to form a white solid (Compound 20).

FIG. 3B illustrates the synthesis scheme of another embodiment of Formula I, II, or III, compound AC620.

Accordingly, 0.5 mol Benzoic acid (Compound 1) and 1 mol triethylamine were dissolved in 400 ml dry dichloromethane. 65 ml chloroacetonitrile was added to the mixture and incubated at room temperature overnight. The triethylamine salt was filtered out under vacuum and the remaining solution was washed twice with 5% Na₂CO₃, 2 N HCl, and water, respectively. The organic layer was separated and dried with MgSO₄. The solvent was removed by vacuum evaporation. The resulting light yellow solution contains benzoxy acetonitrile (Compound 2).

24.39 g benzoxy acetonitrile (Compound 2) and 0.15 mol 1,2,4-trihydroxybenzene were added to 160 ml dry ether cooled in an ice water bath. 8.0 g dry ZnCl₂ was added to the mixture. The mixture was then stirred while adding HCl gas for 2 hr. After yellowish green precipitates were formed, the container was sealed and frozen for 48 hr. Then ether was discarded from the container and 600 ml 50% ethanol was added to the mixture. The reaction mixture was refluxed for 10 hr. Afterwards 140 ml ethanol was distilled from the reaction mixture under vacuum. The mixture was then cooled until yellow precipitates were formed (Compound 11).

14.4 g Compound 11 and 0.125 mol triethylamine were dissolved in 400 ml dry dichloromethane. 0.125 mol T-butyldimethylchorosilane (TBDMSCl) was dissolved in 100 ml dry dichloromethane. The two solutions were mixed for 4 hr. The reaction mixture was then stirred overnight at room temperature. The reaction solution was washed twice with 1% HCl. The organic layer was separated and dried with MgSO₄. The solvent was removed by vacuum evaporation. After a small amount of methanol was added, white precipitates were formed in the mixture (Compound 12).

14.4 g Compound 13 and 0.125 mol triethylamine were dissolved in 400 ml dry dichloromethane. 0.125 mol T-butyldimethylchorosilane (TBDMSCl) was dissolved in 100 ml dry dichloromethane. The two solutions were mixed for 4 hr. The reaction mixture was stirred overnight at room temperature. Then the reaction solution was washed twice with 1% HCl. The organic layer was separated and dried with MgSO₄. The solvent was removed by vacuum evaporation. After a small amount of methanol was added, white precipitates were formed in the mixture (Compound 14.

14.6 g Compound 14 was added to 150 ml H₂O and heated to 70-80° C. with stirring. 0.06 mol KMnO₄ was dissolved in 180 ml H₂O and added to the Compound 14 solution. 10% KOH was added to the mixture to turn the solution alkaline. The solution was then filtered. After the filtrate was cooled, HCl was added to the filtrate to reach pH 2. Then white precipitates were formed in the filtrate (Compound 15).

3.82 g Compound 15 was dissolved in 60 ml dry THF and 0.015 mol SOCl₂ was added to the solution. The reaction mixture was stirred at room temperature overnight. Afterwards, the solvent was removed by vacuum evaporation and white precipitates were formed in the solution (Compound 16).

1 g Compound 12 and 0.9 g Compound 16 were mixed and the mixture was refluxed in 10 ml dry pyridine for 2 hr. The reaction mixture was then poured into a solution consisting of 20 ml crushed ice and 2 ml HCl. The mixture was extracted twice with CH₂Cl₂, and then washed three times with an aqueous phase of Na₂CO₃ and H₂O, respectively. The organic layer was separated and dried with MgSO₄. The solvent was removed by vacuum evaporation. The precipitates were re-crystallized with ethanol to form a white solid (Compound 17).

10 ml THF solution containing 60 mg sodium hydride (washed three times with petroleum ether before use) was added to 10 ml THF solution containing 0.44 g Compound 17. The reaction mixture was refluxed for 4 hr. The reaction mixture was then poured into a mixture consisting of 60 ml ice and 1 ml HCl. The reaction mixture was extracted twice with ether and washed three times with H₂O. The organic layer was separated and dried with MgSO4. The solvent was removed by vacuum evaporation. A yellow solid was obtained after purification by silica gel electrophoresis using an elution buffer of 9% acetone in petroleum ether (Compound 18).

0.34 g KOH was dissolved in 10 ml MeOH and then added to 2.6 g Compound 18. The reaction mixture was refluxed for 2 hr. The reaction mixture was then poured into 100 ml H₂O and H₂SO₄ was used to adjust pH to pH 2. A white solid was formed afterwards (Compound 19).

7.58 g Compound 19 was dissolved in 50 ml dry benzene, and added to 3.3 g Ag₂CO₃. After stirring at room temperature for 1 hr, 7.7 g acetobromo-rutinose was added to the mixture and the reaction mixture was stirred at room temperature overnight. The reaction mixture was then poured into 150 ml aqueous phase of NaHCO₃, and the silver salt was filtered out under vacuum. The reaction mixture was then extracted twice with CHCl₃. The organic layer was separated and dried with MgSO₄. Then 50 ml MeOH and MeONa were added to the reaction mixture and stirred at room temperature overnight. Acetic acid was added to the reaction mixture to bring pH to pH 7. The solvent was removed by vacuum evaporation. A yellow solid was obtained after purification by silica gel electrophoresis using an elution buffer of 20% acetone in petroleum ether (Compound 21).

0.76 g Compound 21 was dissolved in 50 ml dry THF, and added to 0.06 mol tetrabutylammonium fluoride (in 1 M THF) at 0° C. The reaction was complete after 30 min. A yellow solid was obtained after purification by silica gel electrophoresis, which is the purified Compound 22, also known as AC620.

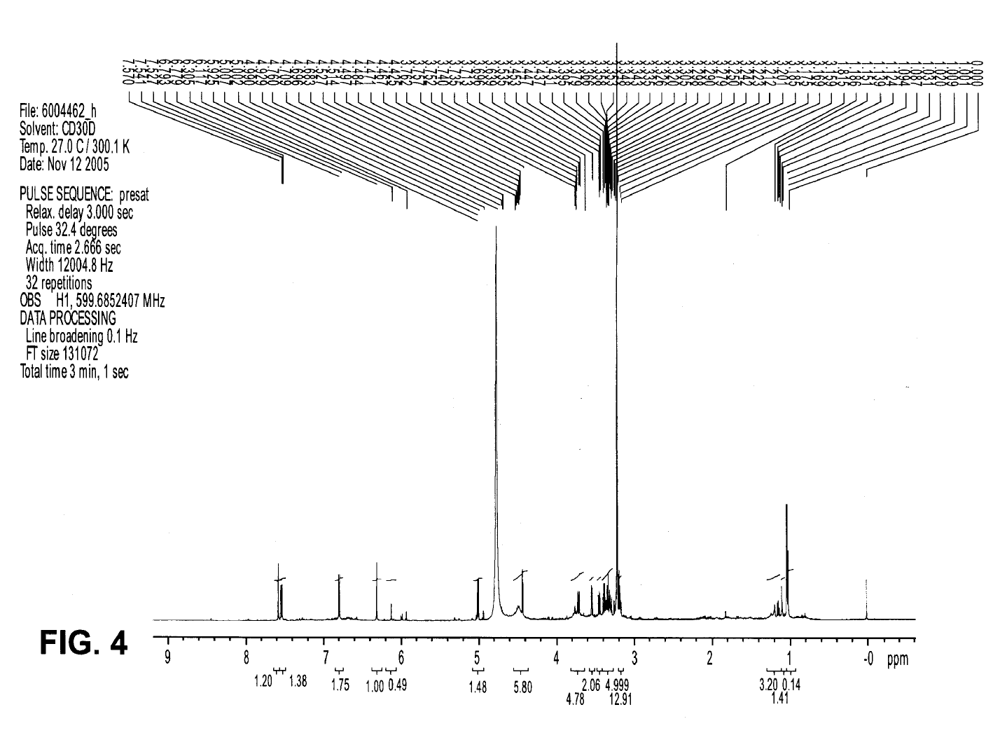

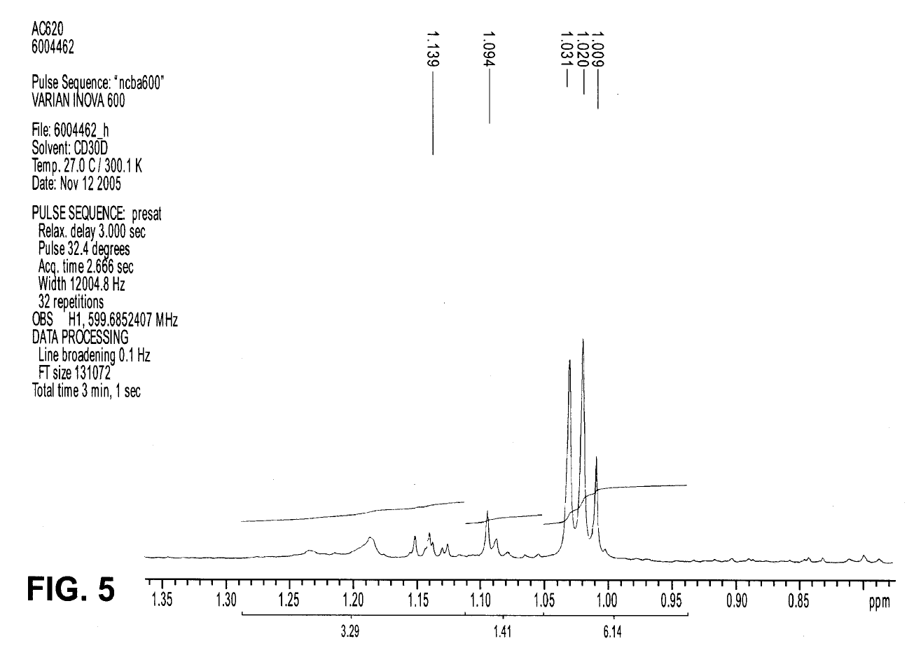

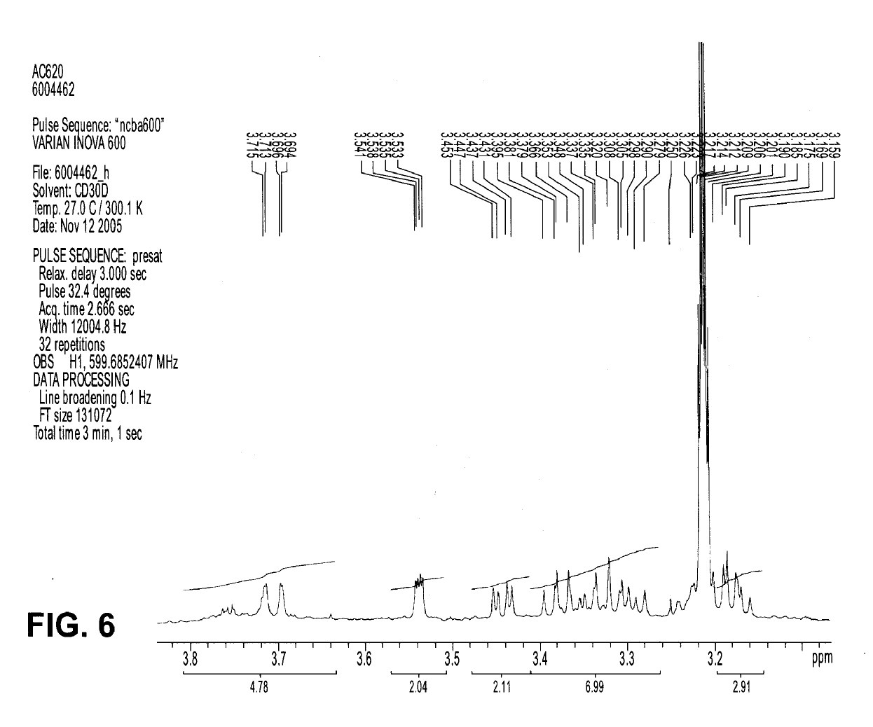

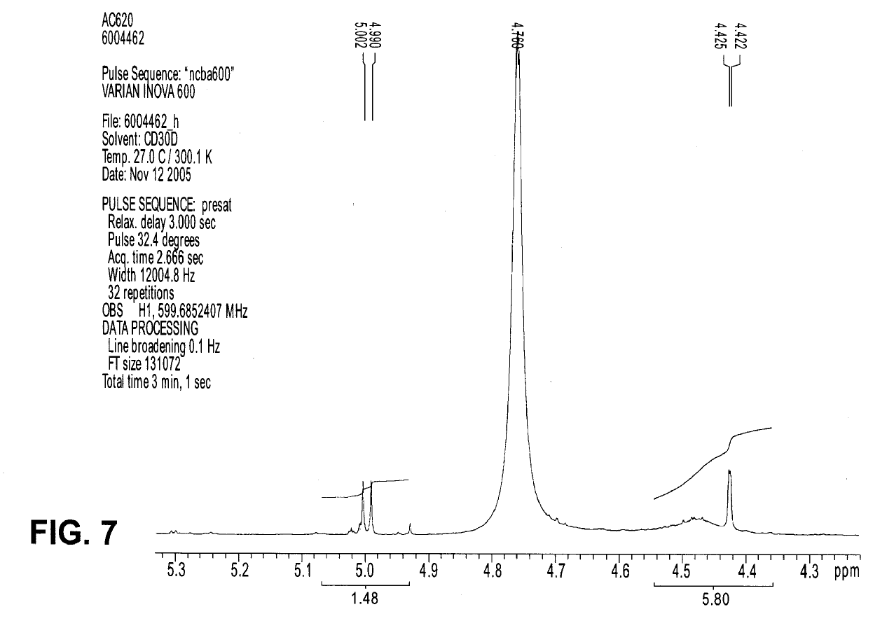

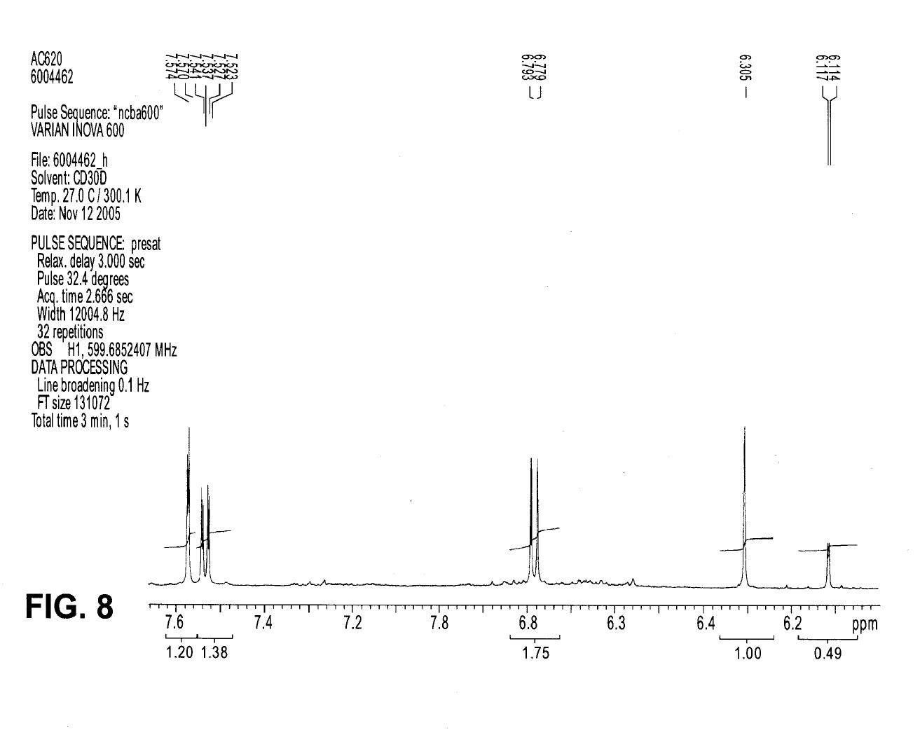

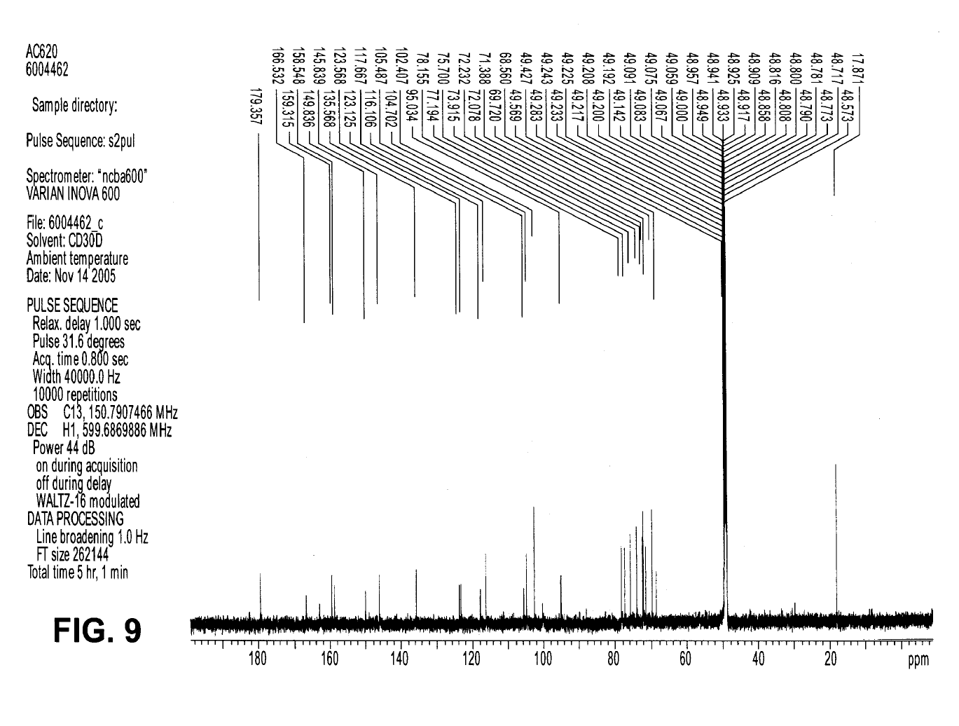

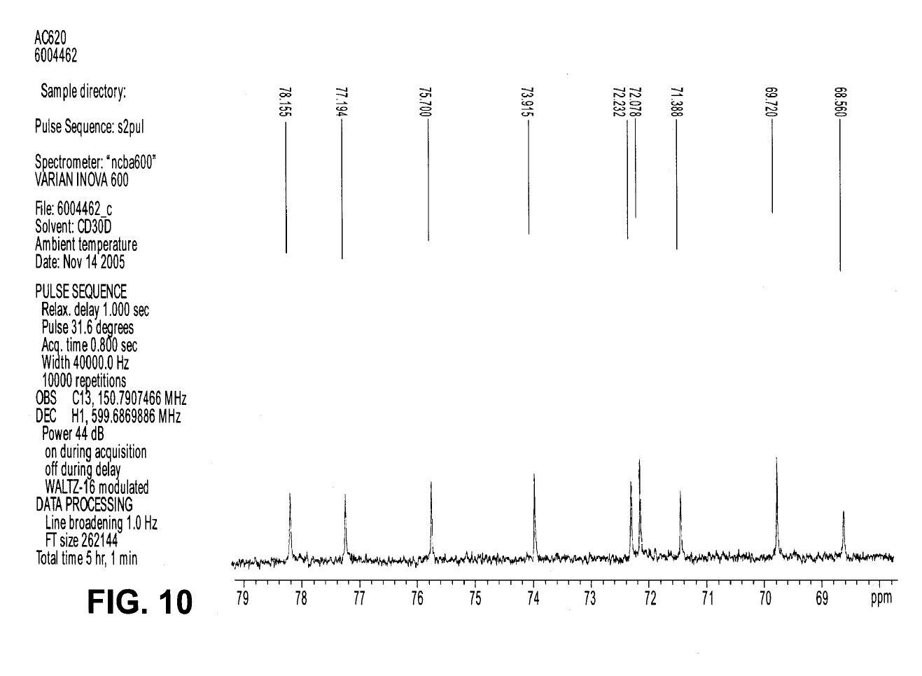

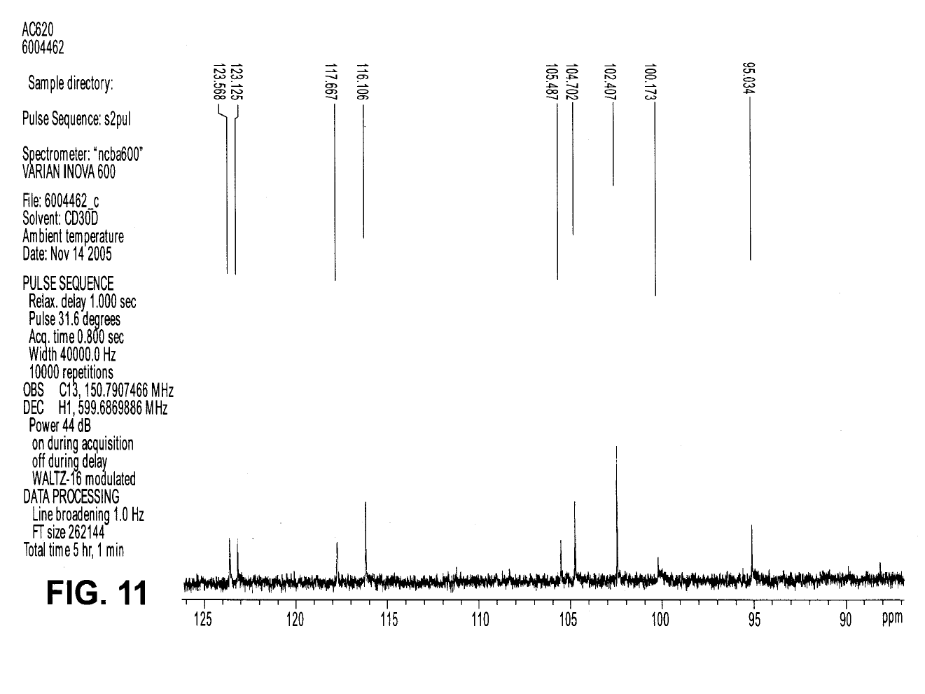

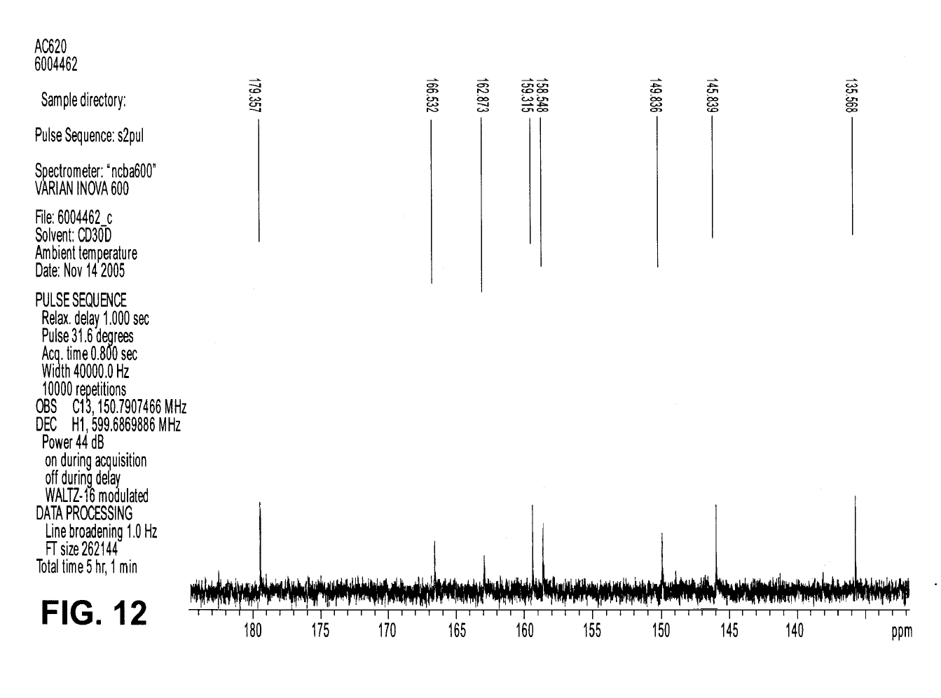

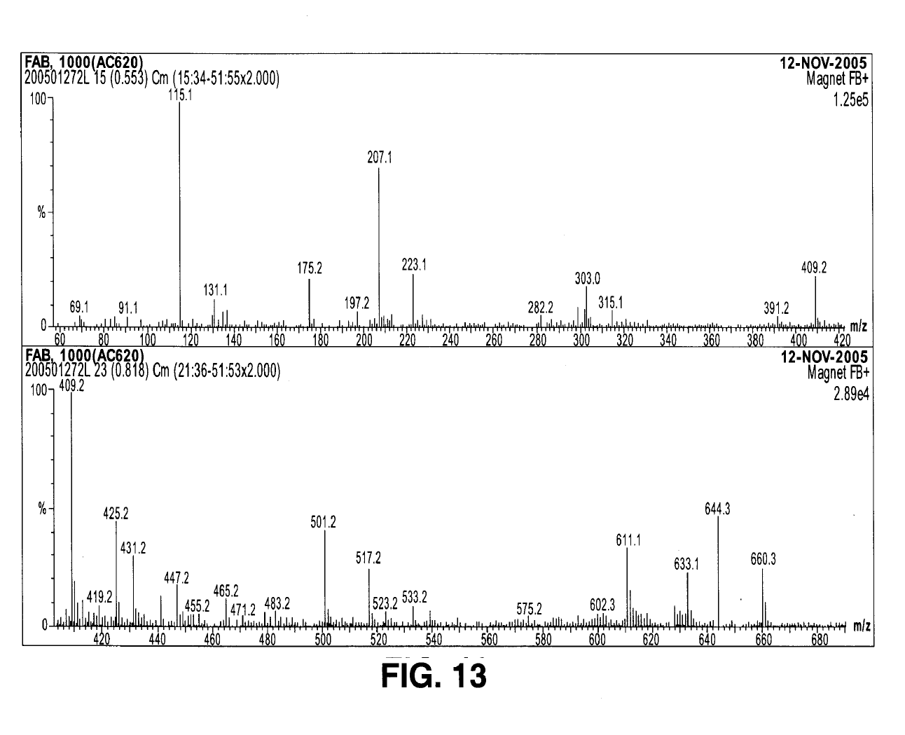

The melting point of AC620 is 188-190° C. NMR and FAB-MS spectroscopy of AC620 are shown in FIGS. 4-12 and 13, respectively.

Compounds of Formula I, II, or III can be prepared by the methods above as individual stereoisomers or as a racemic mixture. Individual stereoisomers of the compounds of Formula I, II, or III can be prepared from racemates by resolution using methods known in the art for the separation of racemic mixtures into their constituent stereoisomers, or using separation of salts of stereoisomers. Compounds of Formula I, II, or III can be isolated in association with solvent molecules by crystallization from, or evaporation of, an appropriate solvent.

The pharmaceutically acceptable acid addition salts of the compound of Formula I, II, or III that contain a basic center can be prepared in a conventional manner. For example, a solution of the free base can be treated with a suitable acid, either neat or in a suitable solution, and the resulting salt isolated either by filtration or by evaporation under vacuum of the reaction solvent. Pharmaceutically acceptable base addition salts can be obtained in an analogous manner by treating a solution of a compound of Formula I, II, or III with a suitable base. Both types of salt can be formed or interconverted using ion-exchange resin techniques. Thus, according to a further aspect of the invention, a method for preparing a compound of Formula I, II, or III, or a salt or solvate (e.g., hydrate) is provided, followed by (i) salt formation, or (ii) solvate (e.g., hydrate) formation.

The following abbreviations are used hereafter in the accompanying examples: hr (hour), g (gram), mol (mole), mmol (millimole), ml (milliliter), MeOH (methanol), HCl (hydrochloric acid), NaCl (sodium chloride).

2. Inhibition of VEGF Expression

AC620 was dissolved in ethanol and then added to cultured DU-145 cells, a hormone-refractory, metastatic prostate cancer cell line. The concentrations tested were 0, 0.1, 0.5, 1 and 4 μM, and the cells were grown for 72 hr. Cells treated with ethanol for the same time period were used as Control. DU-145 cells were maintained in RPMI 1640 medium (PAA Laboratories, Pashing, Austria) supplemented with 10% fetal calf serum (FCS) (Sigma, St. Louis, Mo.). After 72 hr treatment, cell Tysates were collected and immunoblotting was performed. The membranes were probed with primary antibodies against VEGF and actin (Santa Cruz Biotechnology, Santa Cruz, Calif.) followed by horseradish peroxidase (HRP)-conjugated secondary antibodies (Amersham Life Science, Alesbury, U.K.) and visualized using the Enhanced Chemi-Luminescence detection system (ECL) and ECL films (Amersham Pharmacia Biotech).

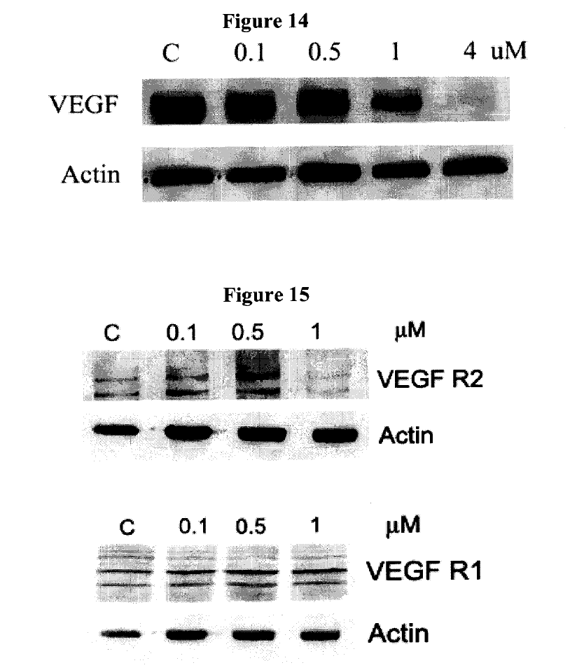

As shown in FIG. 14, cells treated with various concentrations of AC620 showed dose-dependant inhibition of VEGF expression. Treatment with 0.1 μm of AC620 reduced VEGF expression approximately 40%; 0.5 μm reduced VEGF expression approximately 60%; 1 μm reduced VEGF expression approximately 80%; while 4 μm reduced VEGF expression over 95%.

3. Selective Inhibition of VEGF Receptors

DU-145 cells were treated with AC620 at various concentrations (0, 0.1, 0.5, 1 μM) for 72 hr. After 72 hr treatment, cell lysates were collected and immunoblotting was performed. The membranes were probed with primary antibodies against VEGF Receptor I (VEGF R1) and VEGF Receptor II (VEGF R2) and actin (Santa Cruz Biotechnology, Santa Cruz, Calif.) followed by horseradish peroxidase (HRP)-conjugated secondary antibodies and visualized using the Enhanced Chemi-Luminescence detection system (ECL) and ECL films.

As shown in FIG. 15, cells treated with AC620 showed inhibition of VEGF R2 expression, while cells treated with AC620 showed no change in VEGF R1 expression. Treatment with 0.5 μm decreased VEGF R2 expression approximately 10%, while treatment with 1 μm decreased VEGF R2 expression approximately 70%.

4. Inhibition of Cell Proliferation—Dose-Dependant Inhibition

AC620 was incubated with hormone-refractory prostate cancer cell line DU-145 cells and hormone-responsive prostate cancer cell line LNCaP cells at various concentrations (0, 0.1, 0.5, 1, 5 μM) for 72 hr. LNCaP cells were maintained in RPMI 1640 medium (PAA Laboratories, Pashing, Austria) supplemented with 10% fetal calf serum (FCS) (Sigma, St. Louis, Mo.). The effect of AC620 on proliferation of DU-145 cells and LNCaP cells was determined by using a non-radioactive BrdU based cell proliferation assay kit (Roche, Switzerland) according to manufacturer's protocol. BrdU incorporation into the cellular DNA was determined by measuring the absorbance at 450 nm and 690 nm on an ELISA plate reader.

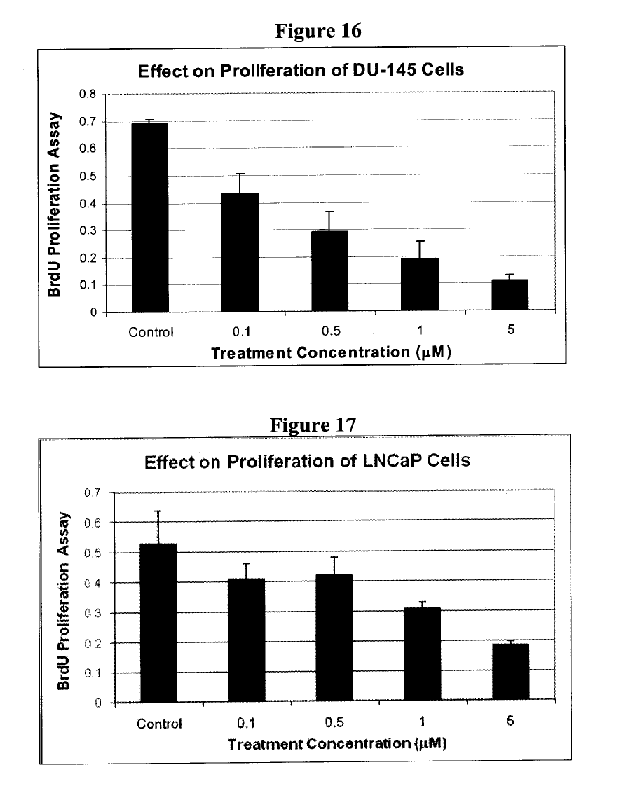

As shown in FIGS. 16 and 17, AC620 significantly inhibited proliferation of both DU-145 cells and LNCaP cells, respectively, and such inhibition is in a dose-dependent manner. The decrease in cell proliferation of DU-145 cells was 37% for 0.1 μm; 58% for 0.5 μm; 72% for 1 μm; and 84% for 5 μm. The decrease in cell proliferation of LNCaP cells was 23% for 0.1 μm; 20% for 0.5 μm; 42% for 1 μm; and 65% for 5 μm.

5. Inhibition of Cell Proliferation—Time-Dependant Inhibition

AC620 was incubated with hormone-refractory prostate cancer cell line DU-145 cells and hormone-responsive prostate cancer cell line LNCaP cells at 0 (Control) and 5 μM for 24, 48 and 72 hr. The effect of AC620 on proliferation of DU-145 cells and LNCaP cells was determined by using a non-radioactive BrdU based cell proliferation assay kit (Roche, Switzerland) according to manufacturer's protocol. BrdU incorporation into the cellular DNA was determined by measuring the absorbance at 450 nm and 690 nm on an ELISA plate reader.

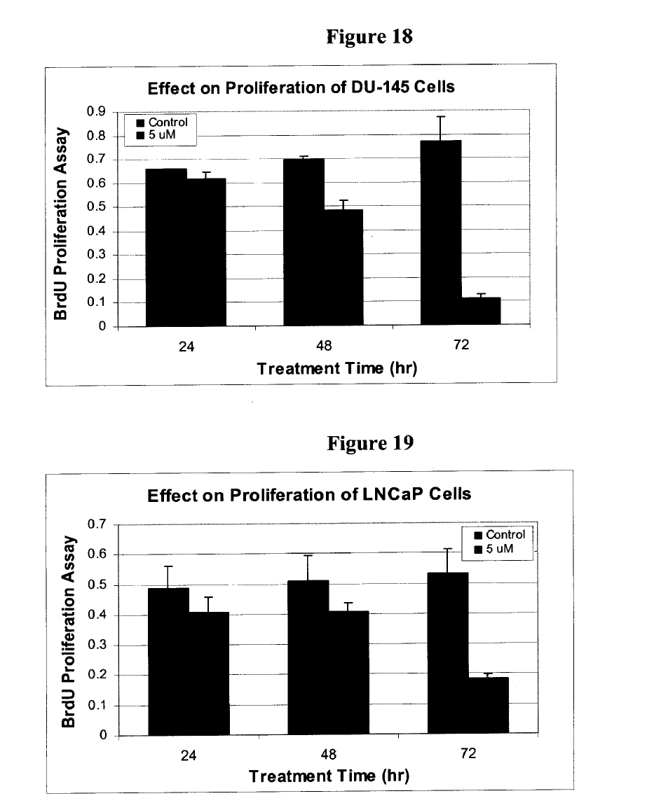

As shown in FIGS. 18 and 19, AC620 significantly inhibited proliferation of both DU-145 cells and LNCaP cells, respectively, and such inhibition is in a time-dependent manner. The inhibition was more profound after 72 hr treatment. The decrease in cell proliferation of DU-145 cells was 6% after 24 hr; 31% after 48 hrs; and 86% after 72 hr. The decrease in cell proliferation of LNCaP cells was 16% after 24 hr; 20% after 48 hr; and 65% after 72 hr.

6. Promotion of Cell Cycle Arrest

AC620 was incubated with DU-145 cells at various concentrations (0, 0.1, 0.5, 1.4 μM) for 72 hr. The cells were then collected and their cell cycle profiles were measured by flow cytometry analysis.

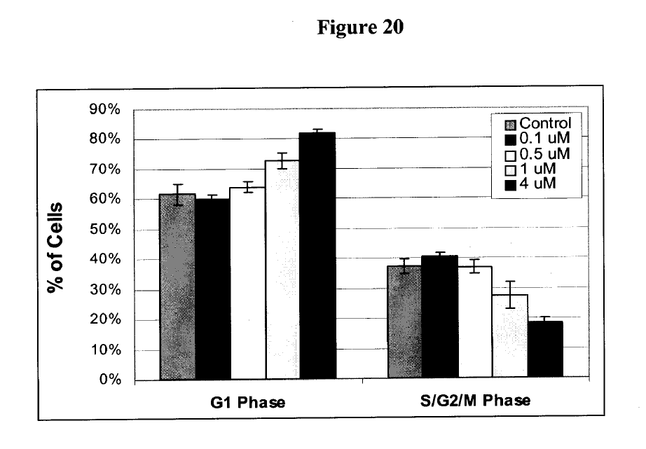

As shown in FIG. 20, treatment of DU-145 cells with AC620 resulted in a pronounced increase in G1 phase cells as compared to Control. The increased number of cells at G1 phase coincided with decreased number of cells in S and G2/M phases. AC620 produced G1 phase cell cycle arrest in a dose-dependent manner with 0% at 0.1 μm, 4% at 0.5 μm; 18% at 1 μm and 32% at 5 μm.

7. Inhibition of Cell Cycle Regulator CDK2 Expression

DU-145 cells were treated with AC620 at various concentrations (0, 0.1, 0.5, 1, 5 μM) for 72 hr. After 72 hr treatment, cell lysates were collected and immunoblotting was performed. The membranes were probed with primary antibodies against CDK2 (Transduction Laboratory, San Jose, Calif.) and actin (Santa Cruz Biotechnology, Santa Cruz, Calif.) followed by horseradish peroxidase (HRP)-conjugated secondary antibodies and visualized using the Enhanced Chemi-Luminescence detection system (ECL) and ECL films.

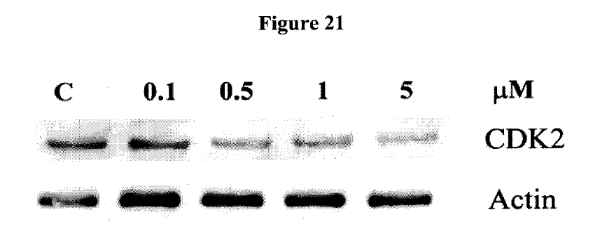

As shown in FIG. 21, cells treated with AC620 showed a dose-dependant inhibition of CDK2 expression with an estimated decrease of CDK2 expression of 0% with 0.1 μm; 10% with 0.5 μm; 20% with 1 μm; and 60% with 4 μm.

8. Promotion of Apoptosis

AC620 was incubated with DU-145 cells and LNCaP cells at various concentrations (0, 0.1, 0.5, 1, 5 μM) for 72 hr. The effect of AC620 on apoptosis of DU-145 cells and LNCaP cells was assessed. The cells were washed in 1×PBS and resuspended in binding buffer (0.01M Hepes pH 7.4, 0.14M NaCl, 2.5 mM CaCl₂), stained with Annexin V conjugated with APC (Pharmingen) and then subjected to flow cytometry analysis using FACS Caliber (BD Biosciences, San Jose, Calif.).

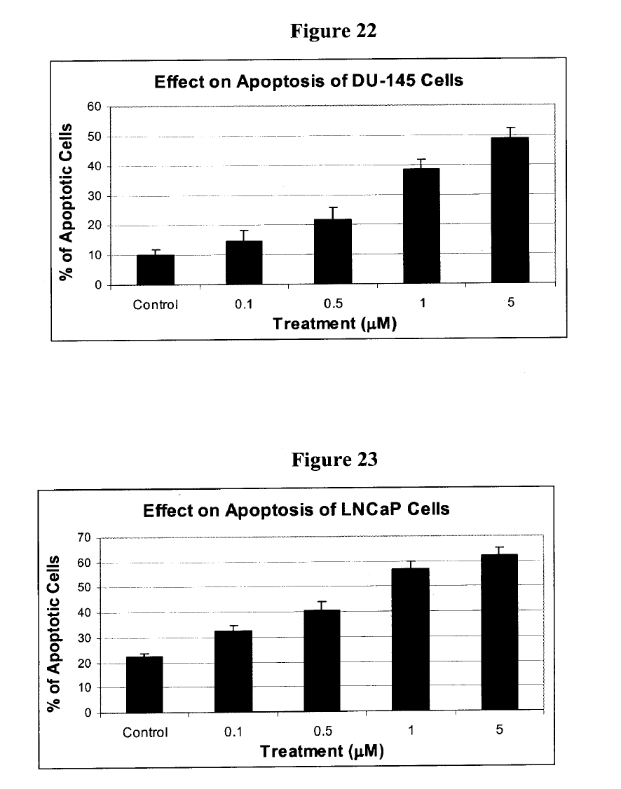

As shown in FIGS. 22 and 23, AC620 significantly increased the level of apoptotic DU-145 and LNCaP cells, respectively, in a dose-dependent manner. For DU-145 cells apoptosis increased 46% with 0.1 μm; 115% with 0.5 μm; 285% with 1 μm; and 389% with 5 μm. For LNCaP cells apoptosis increased 46% with 0.1 μm; 81% with 0.5 μm; 154% with 1 μm; and 177% with 5 μm.

9. Induction of Cancer Cell-Specific Death

AC620 was incubated with DU-145 cells, LNCaP cells and primary human prostate endothelial cells at high concentrations (0, 5, 100, 500, 1000 μM) for 72 hr. The number of viable cells was measured by Annexin-V staining followed by flow cytometry analysis.

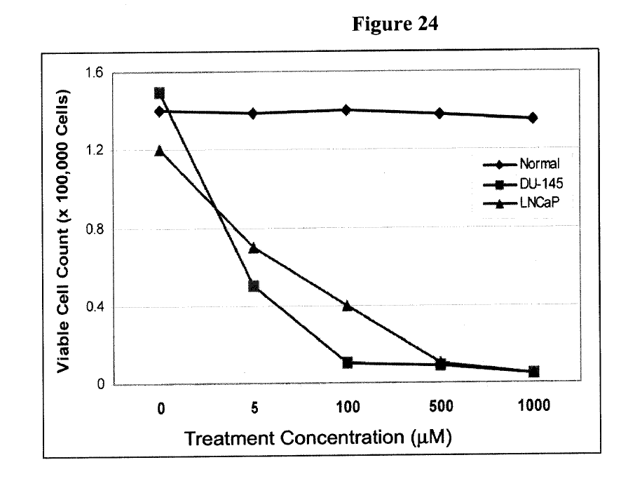

As shown in FIG. 24, there was a marked decrease in viable cancer cells following treatment with AC620. Viable DU-145 cells decreased 67% with exposure to 5 μm; 93% with 100 μm; 94% with 500 μm; and 97% with 1000 μm. Viable LNCaP cells decreased 42% with 5 μm; 67% with 100 μm; 92% with 500 μm; and 96% with 1000 μm. In contrast, primary human prostate endothelial cells remained viable, even when treated with the highest concentration of AC620, 1000 μM. The lack of cell kill in the primary cells demonstrated the cancer cell-specific effect of AC620.

10. Inhibition of Cell Proliferation of Colorectal Cancer Cells

AC620 was incubated with metastatic colorectal cancer cell line HT-29 cells at various concentrations (0, 0.1, 0.5, 1, 4 μM) for 72 hr. The effect of AC620 on proliferation of HT-29 cells was determined by using a non-radioactive, BrdU based cell proliferation assay as described above.

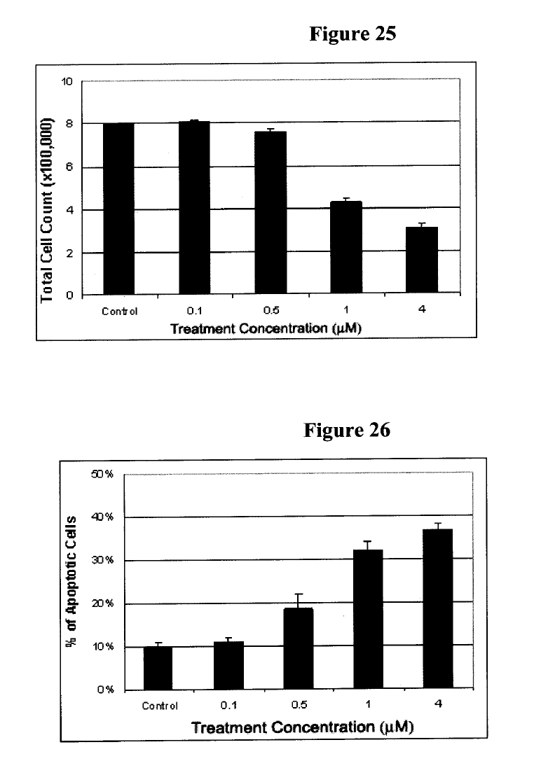

As shown in FIG. 25, AC620 significantly inhibited proliferation of HT-29 cells in a dose-dependent manner. Cell proliferation decreased 0% with 0.1 μm; 5% with 0.5 μm; 46% with 1 μm; and 62% with 4 μm.

11. Promotion of Apoptosis in Colorectal Cancer Cells

AC620 was incubated with metastatic colorectal cancer cell line HT-29 cells at various concentrations (0, 0.1, 0.5, 1, 4 μM) for 72 hr. The effect of AC620 on apoptosis of HT-29 cells was assessed by Annexin V stating as described above.

As shown in FIG. 26, AC620 significantly increased the level of apoptotic HT-29 cells in a dose-dependent manner. Apoptosis increased 10% with 0.1 μm; 87% with 0.5 μm; 220% with 1 μm; and 267% with 5 μm.

12. Inhibition of Tumor Growth in Xenograft Mouse Model

8-12 week old athymic nude mice (Jackson Laboratories, Bar Harbor, Me., US) were randomly assigned to Control (n=10) and Treatment (n=10) groups. Tumor xenografts were generated by injecting DU-145 cells (4×10⁶) subcutaneously into the mice (Day 1). 1 mg/kg body weight of AC620 or PBS (Control) was administrated to the mice by subcutaneous injection starting at the time of tumor implantation (Day 1) and the mice were treated twice a week. Tumors were measured periodically. At Day 30, the mice were sacrificed. Half of each tumor was processed for immunohistochemical analysis and the other half was frozen for subsequent protein and RNA analysis.

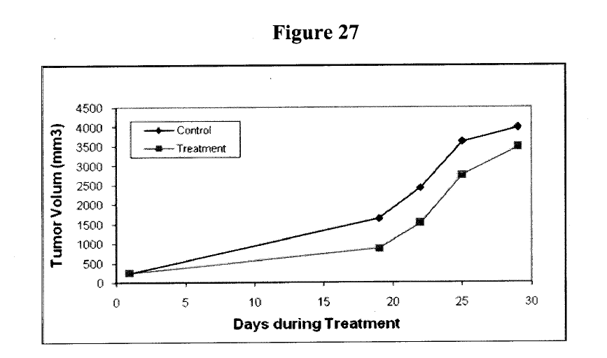

As shown in FIG. 27, as compared to Control, tumor growth in prostate cancer xenograft mouse model was significantly reduced by the treatment of AC620 (P<0.03). The decrease of tumor size was 47% on Day 19; 37% on Day 22; 24% on Day 25; and 13% on Day 29.

13. Inhibition of VEGF and VEGF R2 Expression in Xenograft Mouse Model

Xenograft mouse model was generated and AC620 or PBS (Control) was administrated to the transplanted mice for 30 days as described above. After the mice were sacrificed, immunohistochemical analysis of the tumor samples was performed using VEGF and VEGF R2 antibodies with a semi-automatic system Ventana ES (Ventana Inc, Tucson, Ariz.). The specimens were viewed with an Olympus BX51 microscope. Expression of VEGF in tumor samples at RNA level was measured by RT-PCR.

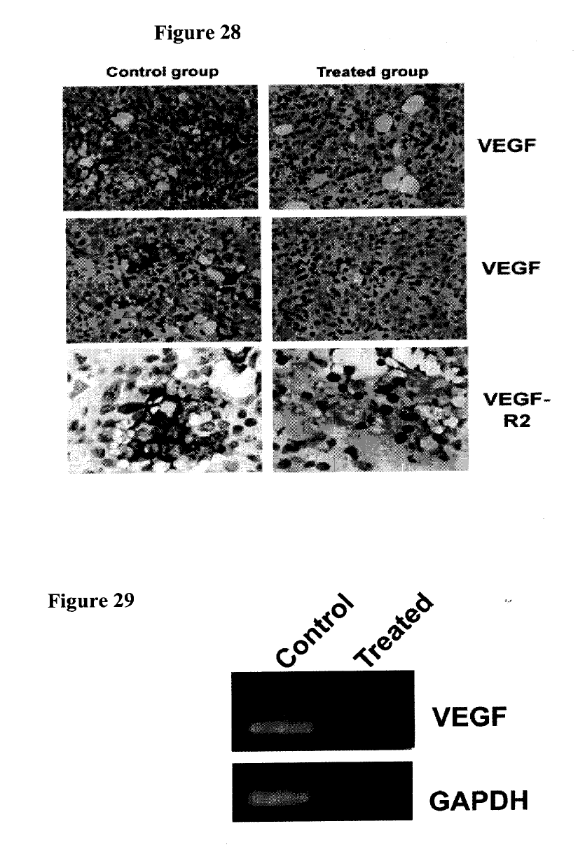

As shown in FIG. 28, the expression of VEGF was completely abolished in tumors treated with AC620 and VEGF R2 expression was significantly reduced. In contrast, high level of expression of both VEGF and VEGF R2 was observed in tumors treated with PBS Control. In addition, samples from the AC620 treatment group showed more differentiated morphology as compared to typical tumor tissue morphology in samples from the Control group.

As shown in FIG. 29, RT-PCR analysis showed an estimated decrease of 90% of VEGF RNA expression level in tumor samples from the AC620 treatment group.

14. Inhibition of CDK1 and CDK2 Expression in Xenograft Mouse Model

Xenograft mouse model was generated and AC620 or PBS (Control) was administrated to the transplanted mice for 30 days as described above. After the mice were sacrificed, immunoblotting analysis of the tumor samples was performed using CDK1 and CDK2 antibodies as described above.

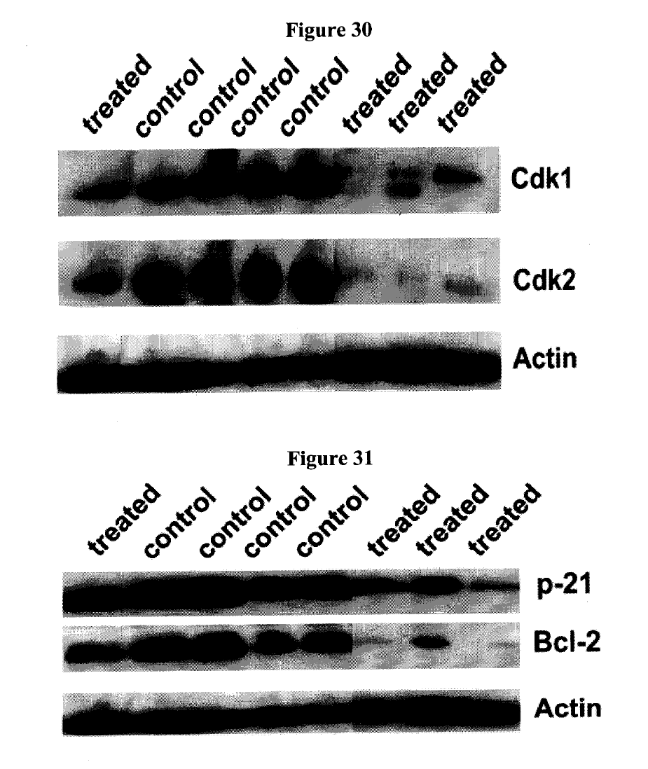

As shown in FIG. 30, as compared to the Control group, both CDK1 and CDK2 expression was significantly reduced or completely abolished in tumor samples from AC620 treatment group. For CDK1 expression, the reduction was approximately 80% for Treatment 1; >95% for Treatment 2; 90% for Treatment 3; and 80% for Treatment 4. For CDK2 expression, the reduction was approximately 80% for Treatment 1; and >95% for Treatments 2, 3 and 4.

15. Inhibition of Bcl-2 and p21 Expression in Xenograft Mouse Model

Xenograft mouse model was generated and AC620 or PBS (Control) was administrated to the transplanted mice for 30 days as described above. After the mice were sacrificed, immunoblotting analysis of the tumor samples was performed using Bcl-2 and p21 antibodies as described above.

As shown in FIG. 31, as compared to the Control group, Bcl-2 expression was significantly reduced or completely abolished in tumor samples from AC620 treatment group with an estimated decrease of 80% for Treatment 1; >95% for Treatment 2; 90% for Treatment 3; and >95% for Treatment 4. p21 expression was also reduced in the AC620 treatment group with 0% for Treatment 1; 70% for Treatment 2; 60% for Treatment 3; and 90% for Treatment 4.

1. A compound of Formula I, its pharmaceutically acceptable salt, ester, prodrug, stereoisomer or tautomer, 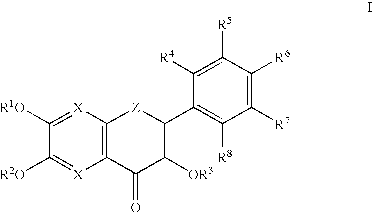
wherein
X is independently CH or N;
Z is independently O, S or NH;
R₁, R₂ and R₃ are independently selected from the group consisting of hydrogen, substituted or unsubstituted C₁-C₁₀ alkyl, substituted or unsubstituted C₁-C₁₀ alkenyl, substituted or unsubstituted C₁-C₁₀ alkynyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether and carbohydrate; and
R₄, R₅, R₆, R₇, R₉ are independently selected from the group consisting of hydrogen, substituted or unsubstituted hydroxyl, substituted or unsubstituted amine, substituted or unsubstituted thiol, substituted or unsubstituted C₁-C₁₀ alkyl, substituted or unsubstituted C₁-C₁₀ alkynyl, substituted or unsubstituted C₁-C₁₀ alkenyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether and carbohydrate.

2. The compound of claim 1, wherein X is CH.

3. The compound of claim 2, wherein Z is O.

4. The compound of claim 3, wherein R₁ and R₂ are independently selected from the group consisting of hydrogen, substituted or unsubstituted C₁-C₁₀ alkyl, substituted or unsubstituted C₁-C₁₀ aliphatic acyl and substituted or unsubstituted C₁-C₁₀ aromatic acyl.

5. The compound of claim 4, wherein R₁ and R₂ are independently selected from the group consisting of hydrogen and substituted or unsubstituted C₁-C₁₀ aliphatic acyl.

6. The compound of claim 5, wherein R₄, R₅, R₆, R₇, R₈ are independently selected from the group consisting of hydrogen, substituted or unsubstituted hydroxyl, substituted or unsubstituted amine, and substituted or unsubstituted C₁-C₁₀ aliphatic acyl.

7. The compound of claims 6, wherein R₄, R₅, R₆, R₇, R₈ are independently selected from the group consisting of substituted or unsubstituted hydroxyl and substituted or unsubstituted amine.

8. The compound of claim 6, wherein at least three of R₄, R₅, R₆, R₇, R₈ are hydrogen.

9. The compound of claim 8, wherein R₅ and R₆ are substituted or unsubstituted hydroxyl and R₄, R₇, R₈ are hydrogen.

10. The compound of claim 9, wherein R₃ is independently selected from the group consisting of hydrogen, substituted or unsubstituted C₁-C₁₀alkyl and carbohydrate.

11. The compound of claim 10, wherein R³ is H or carbohydrate.

12. The compound of claim 11 wherein said carbohydrate is a monosaccharide, a disaccharide, or a polysaccharide.

13. The compound of claim 12, wherein said monosaccharide is selected from the group consisting of glucose, galactose, and fructose.

14. The compound of claim 12, wherein said disaccharide is selected from the group consisting of sucrose, lactose, maltose and rutinose.

15. The compound of claim 12, wherein said polysaccharide is selected from the group consisting of starch, glycogen, and cellulose.

16. A compound of Formula II, its pharmaceutically acceptable salt, ester, prodrug, stereoisomer or tautomer, 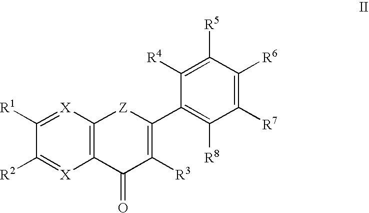
wherein
X is independently CH or N;
Z is independently O, S or NH,
R¹, R², and R³ are each, independently -L-R⁹,
L is —O—, —OC(═O)—, —OP(O)₀₋₁(OR¹⁰)O—, —P(O)₀₋₁(OR¹⁰)O—, —S—, —S(O)—, —S(O)₂—, —S(O)₂NR¹⁰—, or —NR¹⁰—,
R⁹ and R¹⁰ are each independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R¹¹ substituents,
R¹¹ is halogen, —OR², —SH, NH₂, —NR¹²R¹³, —CO₂R¹², —CO₂aryl —C(═O)NR¹²R¹³, —NO₂, —CN, —S(O)₀₋₂C₁₋₁₀alkyl, —S(O)₀₋₂aryl, —SO₂NR¹²R¹³, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR⁹R¹⁰, —SO₂ NR¹²R¹³, or —NR¹²R¹³ substituents,
R⁴, R⁵, R⁶, R⁷ and R⁸ are each independently hydrogen, halogen, —OH, —R¹², —OR², —SH, NH₂, —NR¹²R¹³, —CO₂R¹², —CO₂aryl —C(═O)NR¹²R¹³, —NO₂, —CN, —S(O)₀₋₂C₁₋₁₀alkyl, —S(O)₀₋₂aryl, —SO₂NR¹²R¹³, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR¹²R¹³, —SO₂NR¹²R¹³, —NR¹²R¹³, or trialkylsilyl substituents,
R¹² and R¹³ in each instance, are independently H or unsubstituted or substituted C₁₋₁₀alkyl with one or more aryl, heteroalkyl, heterocyclyl, or hetaryl substituents, wherein each of said alkyl, aryl, heteroalkyl, heterocyclyl, or hetaryl groups is unsubstituted or substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl) substituents.

17. The compound of claim 16, wherein X is CH.

18. The compound of claim 17, wherein Z is O.

19. The compound of claim 18 wherein L is —O—.

20. The compound of claim 19 wherein for R¹, R², and R³, each R⁹ is independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R¹¹ substituents.

21. The compound of claim 20 wherein R⁴, R⁵, R⁶, R⁷ and R⁸ are each independently selected from the group of hydrogen, —OH, —R², —OR¹², NH₂, and —NR¹²R¹³.

22. The compound of claim 21 wherein at least three of R⁴, R⁵, R⁶, R⁷ and R⁸ are hydrogen.

23. The compound of claim 22 wherein R⁴, R⁷ and R⁸ are hydrogen, and R⁵ and R⁶ are each independently selected from the group of hydrogen, —OH and —OR¹².

24. The compound of claim 23 wherein R³ is —OH, —OR¹², or —O-carbohydrate.

25. The compound of claim 23 wherein R³ is —OH or —O-carbohydrate.

26. The compound of claim 25 wherein said carbohydrate is a monosaccharide, a disaccharide, or a polysaccharide.

27. The compound of claim 25, wherein said monosaccharide is selected from the group consisting of glucose, galactose, and fructose.

28. The compound of claim 25, wherein said disaccharide is selected from the group consisting of sucrose, lactose, maltose and rutinose.

29. The compound of claim 25, wherein said polysaccharide is selected from the group consisting of starch, glycogen, and cellulose.

30. A compound of Formula III, its pharmaceutically acceptable salt, ester, prodrug, stereoisomer or tautomer, 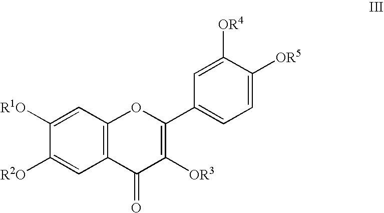
wherein R¹, R², R³, R⁴, and R⁵ are each independently hydrogen, C₁₋₁₀alkyl, C₂₋₁₀alkenyl, C₂₋₁₀alkynyl, aryl, heteroaryl, heterocyclyl, C₃₋₁₀cycloalkyl, or carbohydrate, each of which except for hydrogen is unsubstituted or substituted by one or more independent R⁶ substituents,
R⁶ is halogen, —OR⁷, —SH, NH₂, —NR⁷R⁸, —CO₂R⁷, —CO₂aryl, —C(═O)NR⁷R⁸, —NO₂, —CN, —S(O)₀₋₂C₁₋₁₀alkyl, —S(O)₀₋₂aryl, —SO₂NR⁷R⁸, C₁₋₁₀alkyl, C₂₋₁₀alkenyl, C₂₋₁₀alkynyl, aryl, heteroaryl, heterocyclyl, C₃₋₁₀cycloalkyl, carbohydrate, aryl-C₁₋₁₀alkyl, aryl-C₂₋₁₀alkenyl, aryl-C₂₋₁₀alkynyl, hetaryl-C₁₋₁₀alkyl, hetaryl-C₂₋₁₀alkenyl, hetaryl-C₂₋₁₀alkynyl; each of which is unsubstituted or substituted with one or more independent halo, cyano, nitro, —OC₁₋₁₀alkyl, C₁₋₁₀alkyl, C₂₋₁₀alkenyl, C₂₋₁₀alkynyl, haloC₁₋₁₀alkyl, haloC₂₋₁₀alkenyl, haloC₂₋₁₀alkynyl, —COOH, —C(═O)NR⁷R⁸, —SO₂ NR⁷R⁸, or —NR⁷R⁸ substituents,
R⁷ and R⁸ are independently H or unsubstituted or substituted C₁₋₁₀alkyl with one or more aryl, heteroalkyl, heterocyclyl, or hetaryl substituents, wherein each of said alkyl, aryl, heteroalkyl, heterocyclyl, or hetaryl groups is unsubstituted or substituted with one or more halo, —OH, —C₁₋₁₀alkyl, —CF₃, —O-aryl, —OCF₃, —OC₁₋₁₀alkyl, —NH₂, —N(C₁₋₁₀alkyl)(C₁₋₁₀alkyl), —NH(C₁₋₁₀alkyl), —NH(aryl), —C(O)(C₁₋₁₀alkyl), —C(O)(C₁₋₁₀alkyl-aryl), —C(O)(aryl), —CO₂—C₁₋₁₀alkyl, —CO₂—C₁₋₁₀alkylaryl, —CO₂-aryl, —C(═O)N(C₁₋₁₀alkyl)(C₁₋₁₀alkyl), —C(═O)NH(C₁₋₁₀alkyl), —C(═O)NH₂, —OCF₃, —O(C₁₋₁₀alkyl), —O-aryl, —N(aryl)(C₁₋₁₀alkyl), —NO₂, —CN, —S(O)₀₋₂C₁₋₁₀alkyl, —S(O)₀₋₂C₁₋₁₀alkylaryl, —S(O)₀₋₂ aryl, —SO₂N(aryl), —SO₂N(C₁₋₁₀alkyl)(C₁₋₁₀alkyl), or —SO₂NH(C₁₋₁₀alkyl) or substituents.

31. The compound of claim 30 wherein R³ is hydrogen or carbohydrate.

32. The compound of claim 31 wherein R⁴ and R⁵ are each independently hydrogen, C₁₋₁₀alkyl, or carbohydrate, each of which except for hydrogen is unsubstituted or substituted by one or more independent R⁶ substituents.

33. The compound of claim 32 wherein R¹ and R² are each independently hydrogen, C₁₋₁₀alkyl, or carbohydrate, each of which except for hydrogen is unsubstituted or substituted by one or more independent R⁶ substituents.

34. The compound of claim 33 wherein R¹, R², R⁴, and R⁵ are each independently hydrogen, unsubstituted C₁₋₁₀alkyl, or carbohydrate.

35. The compound of claim 34 wherein R¹, R², R⁴, and R⁵ are each independently hydrogen or unsubstituted C₁₋₁₀alkyl, and R³ is hydrogen or carbohydrate.

36. The compound of claims 35 wherein said carbohydrate is a monosaccharide, a disaccharide, or a polysaccharide.

37. The compound of claim 35, wherein said monosaccharide is selected from the group consisting of glucose, galactose, and fructose.

38. The compound of claim 35, wherein said disaccharide is selected from the group consisting of sucrose, lactose, maltose and rutinose.

39. The compound of claim 35, wherein said polysaccharide is selected from the group consisting of starch, glycogen, and cellulose.

40. The compound of claim 35 wherein R¹, R², R⁴, and R⁵ are each hydrogen, and R³ is hydrogen or carbohydrate.

41. The compound of claim 40 wherein R³ is hydrogen or carbohydrate.

42. The compound of claim 41 wherein R³ is carbohydrate.

43. The compound of claim 42 wherein R³ is rutinose.

44. The compound of Formula III as claimed in claim 30 with the structure:

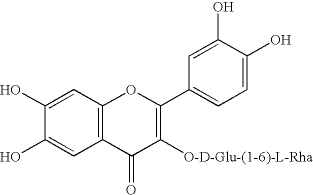

45. A pharmaceutical composition comprising:
a compound of Formula I, II, or III, its pharmaceutically acceptable salt, ester, prodrug, stereoisomer or tautomer; and a pharmaceutically acceptable carrier.

46. The method of treating, reducing the risk of, or preventing cancer in a mammal, comprising: administering to a subject in need thereof an effective amount of a pharmaceutical composition comprising the compound of Formula I, II, or III, its pharmaceutically acceptable salt, ester, prodrug, stereoisomer or tautomer.

47. The method of claim 46, wherein the cancer is selected from the group consisting of adrenal cortical cancer, anal cancer, bile duct cancer, bladder cancer, bone cancer, bone metastasis, adult CNS brain tumors, children CNS brain tumors, breast cancer, Castleman's Disease, cervical cancer, childhood non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, glioblastoma, lymphomas, renal cell cancer, and head and neck cancer.

48. The method of claim 46, further comprising treating the subject with surgery, radiation therapy, chemotherapy, gene therapy, immunotherapy, or a combination thereof.

49. The method of claim 46, wherein the mammal is a human.

50. The method of claim 46, wherein the pharmaceutical composition is administered to the mammal orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, transurethrally, topically, or via an implanted reservoir.

51. A method of treating or preventing a disease in which modulation of angiogenesis is desirable in a mammal comprising administering to a subject in need thereof an effective amount of a pharmaceutical composition comprising the compound of Formula I, II, or III, its pharmaceutically acceptable salt, ester, prodrug, stereoisomer or tautomer.

52. The method of claim 51, wherein the disease in which modulation of angiogenesis is desirable is selected from the group consisting of rheumatoid arthritis, psoriasis, atherosclerosis, diabetic and other retinopathies, retrolentral fibroplasia, neovascular glaucoma, age-related macular degeneration, thyroid hyperplasias, grave's disease, tissue transplantation, chronic inflammation, lung inflammation, nephrotic syndrome, preclampasia, ascites, pericardial effusion, pleural effusion, coronary artery disease, and peripheral artery disease.

53. The method of claim 51, wherein the mammal is a human.

54. The method of claim 51, wherein the pharmaceutical composition is administered to the mammal orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, transurethrally, topically, or via an implanted reservoir.

55. A pharmaceutical composition in unit dosage form comprising, per dosage unit, an amount of a compound of Formula I, II, or III, or a pharmaceutically acceptable salt, ester, prodrug, stereoisomer or tautomer thereof within the range from about 1 mg to about 10 g; and a pharmaceutically acceptable carrier, diluent or excipient.

56. The pharmaceutical composition of claim 55, wherein the amount of a compound of Formula I, II, or III, or a pharmaceutically acceptable salt, ester, prodrug, stereoisomer or tautomer is within the range from about 10 mg to about 500 mg.

57. The pharmaceutical composition of claim 55, wherein the amount of a compound of Formula I, II, or III, or a pharmaceutically acceptable salt, ester, prodrug, stereoisomer or tautomer is within the range from about 10 mg to about 100 mg.

58. A kit, comprising: a container or vessel comprising the compound of Formula I, II, or III, its pharmaceutically acceptable salt, ester, prodrug, stereoisomer or tautomer.

59. The kit of claim 58, wherein the container or vessel comprises a pharmaceutical composition comprising the compound of Formula I, II, or III, its pharmaceutically acceptable salt, ester, prodrug, stereoisomer or tautomer.

60. The kit of claim 59, further comprising: a written instructions for using said pharmaceutical composition for treating or preventing said disease or condition.

61. The kit of claim 60 wherein said disease or condition is selected from the group consisting of adrenal cortical cancer, anal cancer, bile duct cancer, bladder cancer, bone cancer, bone metastasis, adult CNS brain tumors, children CNS brain tumors, breast cancer, Castleman Disease, cervical cancer, Childhood Non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, Hodgkin's disease, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, glioblastoma, lymphomas, renal cell cancer, and head and neck cancer.

62. The kit of claim 60, wherein the disease or condition is selected from the group consisting of, rheumatoid arthritis, psoriasis, atherosclerosis, diabetic and other retinopathies, retrolentral fibroplasia, neovascular glaucoma, age-related macular degeneration, thyroid hyperplasias, grave's disease, tissue transplantation, chronic inflammation, lung inflammation, nephrotic syndrome, preclampasia, ascites, pericardial effusion, pleural effusion, coronary artery disease, and peripheral artery disease.
